(19) United States
(12) Patent Application Publication
(10) Pub. No.: US 2020/0237834 A1
(43) Pub. Date: Jul. 30, 2020

(54) COMPOSITIONS COMPRISING BACTERIAL STRAINS

(71) Applicant: 4D Pharma Research Limited, Aberdeen (GB)

(72) Inventors: Imke Elisabeth MULDER, Aberdeen (GB); Seanin Marie MCCLUSKEY, Aberdeen (GB); Helene SAVIGNAC, Aberdeen (GB); Ian JEFFERY, Cork (IE)

(21) Appl. No.: 16/784,958

(22) Filed: Feb. 7, 2020

Related U.S. Application Data
(63) Continuation of application No. PCT/EP2018/071831, filed on Aug. 10, 2018.

(30) Foreign Application Priority Data
Aug. 10, 2017 (GB) 1712857.0
Jan. 19, 2018 (GB) 1800866.4
Jul. 16, 2018 (EP) 18183642.0

(51) Int. Cl. A61K 35/74; A61K 9/48

(52) U.S. Cl. CPC A61K 35/74; A61K 9/0053; A61K 9/4808

(57) ABSTRACT

The invention provides compositions comprising bacterial strains and the use of such compositions in the treatment of disease.

### CROSS-REFERENCE

This application is a continuation of International Application No. PCT/EP2018/071831, filed Aug. 10, 2018, which claims the benefit of Great Britain Application No. 1712857.0, filed Aug. 10, 2017, Great Britain Application No. 1800866.4, filed Jan. 19, 2018, and European Application No. 18183642.0, filed Jul. 16, 2018, all of which are hereby incorporated by reference in their entirety.

### SEQUENCE LISTING

The instant application contains a Sequence Listing which has been submitted electronically in ASCII format and is hereby incorporated by reference in its entirety. Said ASCII copy, created on Feb. 4, 2020, is named 56708-731_301_SL.txt and is 4,128,360 bytes in size.

### TECHNICAL FIELD

This invention is in the field of compositions comprising bacterial strains and the use of such compositions in the treatment of disease.

### BACKGROUND TO THE INVENTION

The human intestine is thought to be sterile in utero, but it is exposed to a large variety of maternal and environmental microbes immediately after birth. Thereafter, a dynamic period of microbial colonization and succession occurs, which is influenced by factors such as delivery mode, environment, diet and host genotype, all of which impact upon the composition of the gut microbiota, particularly during early life. Subsequently, the microbiota stabilizes and becomes adult-like [1]. The human gut microbiota contains more than 500-1000 different phylotypes belonging essentially to two major bacterial divisions, the Bacteroidetes and the Firmicutes [2]. The successful symbiotic relationships arising from bacterial colonization of the human gut have yielded a wide variety of metabolic, structural, protective and other beneficial functions. The enhanced metabolic activities of the colonized gut ensure that otherwise indigestible dietary components are degraded with release of by-products providing an important nutrient source for the host. Similarly, the immunological importance of the gut microbiota is well-recognized and is exemplified in germfree animals which have an impaired immune system that is functionally reconstituted following the introduction of commensal bacteria [3-5].

Dramatic changes in microbiota composition have been documented in gastrointestinal disorders such as inflammatory bowel disease (IBD). For example, the levels of Clostridium cluster XIVa bacteria are reduced in IBD patients whilst numbers of E. coli are increased, suggesting a shift in the balance of symbionts and pathobionts within the gut [6-9]. Interestingly, this microbial alteration associated with the IBD inflammatory state is characterised by imbalances in T effector cell populations.

A hallmark of many human diseases linked to microbiota alteration is loss of microbiota diversity, distinct from so-called dysbiosis which is simply an altered microbiota composition compared to the typical aggregate microbiota in healthy subjects. Changes in the diversity of the gut microbiota have been linked to modulation in the risk of developing cancer [10]. Re-establishing the healthy microbiota can be difficult as the bacteria in the gut are resistant to colonisation. This poses a challenge when trying to treat the microbiota of unhealthy subjects by increasing the diversity of the microbiota [11]. However, the links between microbiota diversity and cancer are not well understood.

There is a requirement in the art for new methods of treating diseases which benefit from an increase in microbiota diversity and/or increased stability of the microbiota diversity.

### SUMMARY OF THE INVENTION

The inventors have developed new therapies for treating and/or preventing diseases by increasing and/or stabilising the intestinal microbiota diversity in a subject. In particular, the inventors have found that bacterial strains from the genus Enterococcus can be effective in increasing and/or stabilising the intestinal microbiota diversity in the distal gut of a subject. The inventors have identified that bacterial strains of the species selected from the list consisting of Enterococcus gallinarum and Enterococcus caselliflavus can be particularly effective at increasing and/or stabilising the microbiota diversity in a subject, especially a subject diagnosed with a disease.

The invention provides a composition comprising a bacterial strain of the genus Enterococcus, for use in a method of increasing and/or stabilising the microbiota diversity in a subject. Similarly, the invention also provides a method of increasing and/or stabilising the microbiota diversity in a subject wherein the method comprises a step of administering a composition comprising a bacterial strain of the genus Enterococcus to a subject. Preferably, the Enterococcus is a bacterial strain of the species Enterococcus gallinarum or Enterococcus caselliflavus.

The expression “increasing the microbiota diversity” is used herein to mean increasing the number of different types of bacteria and/or the evenness of the different types of bacteria in the microbiota of a subject. The microbiota diversity can be measured by an increase in the number of different genera, species or strains of bacteria in a subject. This increase in microbiota diversity can be in the intestine of the subject or in the distal gut of the subject. The increase or evenness may be measured relative to the diversity/evenness in the subject before administration of a composition according to the invention. The relative abundance of the different types of bacteria in the microbiota becomes more even following treatment with a composition of the invention.

By “stabilising microbiota diversity” it is meant that the relative numbers of the different genera in the microbiota remain stable (e.g. they fluctuate no more than 70%, 80%, 90% 95% or 99% between two measurements). The stabilisation of microbiota diversity can be in the intestine of the subject or in the distal gut of the subject. The relative stability may be assessed relative to the stability before a composition according to the invention has been administered.

The stability of a subject's microbiota can be assessed by comparing the microbiome from the subject at two different time points. The two different time points can be at least three days apart or more, for example at least 1 week, 2 weeks, 1 month, 3 months, 6 months, 1 year, or 2 years apart. The two different time points may be 3-7 days apart, 1-2 weeks apart, 2-4 weeks apart, 4-8 weeks apart, 8-24 weeks apart, 24-40 weeks apart, 40-52 weeks apart or more than 52 weeks apart. More than two different time points can be used, e.g. three, four, five or more than five time points. Suitable intervals are chosen between the various time points, for example, as set out above.

The increase or stabilisation in microbiota diversity may be quantified by measuring the number of the sequence-based bacterial classifications or Operational Taxonomic Units (OTUs) in a sample, typically determined by 16S rRNA amplicon sequencing methods. An increase of diversity may be measured by an increase in the Shannon Diversity Index, or the Chao index [12].

The inventors have also developed new therapies for treating and preventing diseases by increasing the microbiota diversity and/or stabilising the microbiota diversity in a subject. For example, the invention provides compositions comprising a bacterial strain of the genus Enterococcus for use in a method of increasing the microbiota diversity and/or stabilising the microbiota diversity in a subject diagnosed with cancer. As those skilled in the art will recognise, cancer patients, as a result of the effects of the disease and/or of their treatment may suffer from a reduction in the diversity of their microbiome which may be linked to the development or exacerbation of secondary diseases.

A reduction of diversity of the microbiome has been implicated in the development and/or exacerbation of an increasing number of diseases. These include neurological conditions such as Alzheimer's disease [13], Parkinson's disease [14], autism [15] and multiple sclerosis [16,17]; gastrointestinal disorders such as irritable bowel syndrome [18] and inflammatory bowel disease [19,20,21]; musculoskeletal disorders such as rheumatoid arthritis [22] and psoriatic arthritis [23]; metabolic disorders including Type I diabetes [24]; and wasting/fatigue conditions including sarcopenia [25] and myalgic encephalomyelitis [26].

Thus, the compositions of the present invention which can stabilise or improve the microbiome diversity of subjects (including in cancer patients) and thus treat or prevent diseases characterised by reduced microbiome diversity is desirable.

The invention provides a composition comprising a bacterial strain of the genus Enterococcus, for use in a method of increasing the microbiota diversity and/or stabilising the microbiota diversity in a subject. Bacteria from the genus Enterococcus can be identified by using a biochemical key [27]. A bacterial strain of the composition may have a 16S rRNA sequence that is at least 95% identical to the 16S rRNA sequence of a bacterial strain of Enterococcus gallinarum. The invention thus provides a composition comprising a bacterial strain that has a 16S rRNA sequence that is at least 95% identical to SEQ ID NO: 1, 2 or 5 (over 100% of the sequence) for use in a method of increasing the microbiota diversity and/or the stability of the microbiota in a subject.

The bacterial strain in the composition may be of Enterococcus gallinarum or Enterococcus caselliflavus. Closely related strains may also be used, such as bacterial strains that have a 16S rRNA sequence that is at least 95%, 96%, 97%, 98%, 99%, 99.5% or 99.9% identical to the 16S rRNA sequence of a bacterial strain of Enterococcus gallinarum or Enterococcus caselliflavus. Preferably, the bacterial strain has a 16S rRNA sequence that is at least 95%, 96%, 97%, 98%, 99%, 99.5% or 99.9% identical to SEQ ID NO:1, 2 or 5. Preferably, the bacterial strain for use in the invention has the 16S rRNA sequence represented by SEQ ID Nos 1, 2 or 5. This is preferred as the inventors have found that such a strain increases the microbiota diversity particularly well.

The bacteria strain may be the Enterococcus gallinarum bacterium deposited under accession number NCIMB 42488. The bacteria strain may be the Enterococcus gallinarum bacterium deposited under accession number NCIMB 42761. The bacteria strain may be an Enterococcus caselliflavus bacterium.

The composition of the invention may be suitable for oral administration. Oral administration of the strains of the composition can be effective for increasing the microbiota diversity in a subject. Also, oral administration is convenient for patients and practitioners and allows delivery to and/or partial or total colonisation of the intestine.

The composition of the invention may comprise one or more pharmaceutically acceptable excipients or carriers. The composition of the invention may comprise a bacterial strain that has been lyophilised. Lyophilisation is an effective and convenient technique for preparing stable compositions that allow delivery of bacteria.

The invention provides a food product comprising a composition as described above. The invention provides a vaccine composition comprising a composition as described above.

The invention also provides the use in therapy of a combination of a composition comprising a bacterial strain of the genus Enterococcus (preferably of the species Enterococcus gallinarum) and cyclophosphamide.

Additionally, the invention provides a method of increasing the microbiota diversity in a subject comprising administering a composition comprising a bacterial strain of the species Enterococcus gallinarum to the subject.

### BRIEF DESCRIPTION OF DRAWINGS

FIG. 1A: The Observed diversity after treatment with MRX518 at time points Day-14, Day 0 and Day 22 in EMT6 mice.

FIG. 1B: The Shannon diversity after treatment with MRX518 at time points Day-14, Day 0 and Day 22 in EMT6 mice.

FIG. 1C: The Observed diversity after treatment with MRX0554 at time points Day-14, Day 0 and Day 22 in EMT6 mice.

FIG. 1D: The Shannon diversity after treatment with MRX0554 at time points Day-14, Day 0 and Day 22 in EMT6 mice.

FIG. 1E: The Observed diversity after treatment with MRX0858 at time points Day-14, Day 0 and Day 22 in EMT6 mice.

FIG. 1F: The Shannon diversity after treatment with MRX0858 at time points Day-14, Day 0 and Day 22 in EMT6 mice.

FIG. 1G: The Observed diversity after treatment with REF 10 at time points Day-14, Day 0 and Day 22 in EMT6 mice.

FIG. 1H: The Shannon diversity after treatment with REF 10 at time points Day-14, Day 0 and Day 22 in EMT6 mice.

FIG. 1I: The Observed diversity after treatment with Anti-CTLA4 at time points Day-14, Day 0 and Day 22 in EMT6 mice.

FIG. 1J: The Shannon diversity after treatment with Anti-CTLA4 at time points Day-14, Day 0 and Day 22 in EMT6 mice.

FIG. 2: The Shannon diversity at Day 18 after treatment with bacterial strains MRX518 (G2), MRX0554 (G3), MRX0858 (G4), REF10-DSM100110 (G5), Anti-CTLA4 (G6) and untreated (G1) in LLC mice.

FIGS. 3A-3C: (FIG. 3A) PCoA ordination plot of microbiota profiles based on Bray-Curtis dissimilarities. The data is grouped according to timepoint and includes all treatment groups (p-value=0.001). Total N=210; Each timepoint n=70; (FIG. 3B) PCoA ordination plot of microbiota profiles based on Bray-Curtis dissimilarities. The data is grouped according to treatment at timepoint D-15. (p-value=0.077). Total N=70; Each treatment n=10. (FIG. 3C) PCoA ordination plot of microbiota profiles based on Bray-Curtis dissimilarities. The data is grouped according to treatment at timepoint D22. (p-value=0.001). Total N=70; Each treatment n=10.

### DISCLOSURE OF THE INVENTION

### Bacterial Strains

The compositions for use according to the invention comprise a bacterial strain of the genus Enterococcus. The examples demonstrate that bacteria of this genus are useful for increasing and/or stabilising the microbiota diversity in a subject. The preferred bacterial species of the genus are Enterococcus gallinarum or Enterococcus caselliflavus. Bacterial strains of Enterococcus gallinarum deposited under accession numbers NCIMB 42488 and NCIMB 42761 are preferred as the inventors have seen good results with these strains. It is preferred that the bacterial strain is not Enterococcus hirae.

Enterococcus spp. can be identified by random amplification of polymorphic DNA (RAPD) analysis. RAPD analysis does not require any specific knowledge of the DNA sequence of the target organism. RAPD markers are DNA fragments from PCR amplification of random segments of genomic DNA with a single primer of arbitrary nucleotide sequence and which are able to differentiate between genetically distinct individuals [28].

Enterococcus gallinarum forms coccoid cells, mostly in pairs or short chains. It is nonmotile and colonies on blood agar or nutrient agar are circular and smooth. Enterococcus gallinarum reacts with Lancefield group D antisera. The type strain of Enterococcus gallinarum is F87/276=PB21=ATCC 49573=CCUG 18658=CIP 103013=JCM 8728=LMG 13129=NBRC 100675=NCIMB 702313 (formerly NCDO 2313)=NCTC 12359 [29]. The GenBank accession number for a 16S rRNA gene sequence of Enterococcus gallinarum is AF039900 (disclosed herein as SEQ ID NO:1). An exemplary Enterococcus gallinarum strain is described in [29].

The Enterococcus gallinarum strain deposited under accession number NCIMB 42488 was tested in the Examples and is also referred to herein as strain MRX518. A 16S rRNA sequence for the MRX518 strain that was tested is provided in SEQ ID NO:2. Strain MRX518 was deposited with the international depositary authority NCIMB, Ltd. (Ferguson Building, Aberdeen, AB21 9YA, Scotland) by 4D Pharma Research Ltd. (Life Sciences Innovation Building, Aberdeen, AB25 2ZS, Scotland) on 16th November 2015 as “Enterococcus sp” and was assigned accession number NCIMB 42488.

The Enterococcus gallinarum strain deposited under accession number NCIMB 42761 was tested in the Examples and is also referred to herein as strain MRX554. References to MRX554 and MRx0554 are used interchangeably. Strain MRX554 was deposited with the international depositary authority NCIMB, Ltd. (Ferguson Building, Aberdeen, AB21 9YA, Scotland) by 4D Pharma Research Ltd. (Life Sciences Innovation Building, Aberdeen, AB25 2ZS, Scotland) on 22 May 2017 as “Enterococcus sp” and was assigned accession number NCIMB 42761.

The genome of the Enterococcus gallinarum strain MRX518 comprises a chromosome and plasmid. A chromosome sequence for strain MRX518 is provided in SEQ ID NO:3. A plasmid sequence for strain MRX518 is provided in SEQ ID NO:4. These sequences were generated using the PacBio RS II platform.

Bacterial strains closely related to the strains tested in the examples are also expected to be effective for increasing and/or stabilising the microbiota diversity in a subject. A composition according to the invention may thus comprise a strain which can increase and/or stabilise the microbial diversity in a subject relative to the level or stability of the microbial diversity in the subject before the composition was administered.

The bacterial strain for use in the invention can have a 16S rRNA sequence that is at least 95%, 96%, 97%, 98%, 99%, 99.5% or 99.9%, preferably at least 99.5% or 99.9%, identical to the 16S rRNA sequence of a bacterial strain of Enterococcus gallinarum or Enterococcus caselliflavus. The bacterial strain for use in the invention may have a 16S rRNA sequence that is at least 95%, 96%, 97%, 98%, 99%, 99.5% or 99.9% identical to SEQ ID NO:1, 2, or 5. Preferably, the bacterial strain for use in the invention has the 16S rRNA sequence represented by SEQ ID NOs: 1, 2 or 5.

Bacterial strains that are biotypes of the bacterium deposited under accession number NCIMB 42488, or NCIMB 42761 are also expected to be effective for increasing and/or stabilising the microbiota diversity in a subject. A biotype is a closely related strain that has the same or very similar physiological and biochemical characteristics, e.g. it can increase and/or stabilise the microbial diversity in a subject relative to the microbial diversity in the subject before the composition was administered to the same or similar level (e.g. x±20%, x±10%, x±5%, or x±1%) as a bacterium deposited under accession number NCIMB 42488, or NCIMB 42761.

Strains that are biotypes of the bacterium deposited under accession number NCIMB 42488 or NCIMB 42761 and that are suitable for use in the invention may be identified by sequencing other nucleotide sequences for the bacterium deposited under accession number NCIMB 42488 or NCIMB 42761. For example, substantially the whole genome may be sequenced and a biotype strain for use in the invention may have at least 95%, 96%, 97%, 98%, 99%, 99.5% or 99.9% sequence identity across at least 80% of its whole genome (e.g. across at least 85%, 90%, 95% or 99%, or across its whole genome). For example a biotype strain can have at least 98% sequence identity across at least 98% of its genome or at least 99% sequence identity across 99% of its genome. Other suitable sequences for use in identifying biotype strains may include hsp60 or repetitive sequences such as BOX, ERIC, (GTG)₅ (SEQ ID NO: 6), or REP [30].

A biotype strain may have a 16S rRNA sequence with at least 95%, 96%, 97%, 98%, 99%, 99.5% or 99.9% sequence identity to the corresponding 16S rRNA sequence of the bacterium deposited under accession number NCIMB 42488. A biotype strain can have a 16S rRNA sequence with at least 95%, 96%, 97%, 98%, 99%, 99.5% or 99.9% sequence identity to the corresponding 16S rRNA sequence of strain MRX518 deposited as NCIMB 42488 and comprises a 16S rRNA sequence that is at least 99% identical (e.g. at least 99.5% or at least 99.9% identical) to SEQ ID NO:2. A biotype strain can have a 16S rRNA sequence with at least 95%, 96%, 97%, 98%, 99%, 99.5% or 99.9% sequence identity to the corresponding 16S rRNA sequence of strain MRX518 deposited as NCIMB 42488 and has the 16S rRNA sequence of SEQ ID NO:2. Preferably, a biotype strain has a 16S rRNA sequence which is at least 99% identical (e.g. at least 99.5% identical or at least 99.9% identical) to the corresponding sequence of strain MRX518 deposited as NCIMB 42488.

A biotype strain may have a whole genome sequence with at least 95%, 96%, 97%, 98%, 99%, 99.5% or 99.9% sequence identity to the corresponding whole genome sequence of the bacterium deposited under accession number NCIMB 42761. A biotype strain can have a whole genome sequence with at least 95%, 96%, 97%, 98%, 99%, 99.5% or 99.9% sequence identity to the corresponding whole genome sequence of strain MRX554 deposited as NCIMB 42761 and further comprise a 16S rRNA sequence that is at least 99% identical (e.g. at least 99.5% or at least 99.9% identical) to SEQ ID NO:5. A biotype strain can have a whole genome sequence with at least 95%, 96%, 97%, 98%, 99%, 99.5% or 99.9% sequence identity to the corresponding sequence of strain MRX518 deposited as NCIMB 42488 and has the 16S rRNA sequence of SEQ ID NO:5. Preferably, a biotype strain has a whole genome sequence which is at least 99% identical (e.g. at least 99.5% identical or at least 99.9% identical) to the corresponding sequence of strain MRX554 deposited as NCIMB 42761.

A biotype of the invention will have a similar efficacy in increasing and/or stabilising the intestinal microbiota diversity in a subject as Enterococcus gallinarum or Enterococcus caselliflavus, as defined above. Thus, the biotype will effect an increase which is at least 80%, at least 85%, at least 90%, at least 95% or at least 99% of the increase compared to the increase effected by a bacterium of the strain Enterococcus gallinarum or Enterococcus caselliflavus as defined above. Alternatively, or in addition, a biotype of the invention may stabilise the microbiota diversity to a similar level as Enterococcus gallinarum or Enterococcus caselliflavus, as defined above, i.e. it may maintain a number of different types of bacteria in the microbiota of a subject which is at least 80%, at least 85%, at least 90%, at least 95% or at least 99% of the of the number of different types of bacteria stabilised by Enterococcus gallinarum or Enterococcus caselliflavus as defined above. The microbial diversity can be assessed by considering the number of different types of bacteria.

The bacterial strain for use in the invention may have a chromosome with sequence identity to SEQ ID NO:3. The bacterial strain for use in the invention can have a chromosome with at least 90% sequence identity (e.g. at least 92%, 94%, 95%, 96%, 97%, 98%, 99% or 100% sequence identity) to SEQ ID NO:3 across at least 60% (e.g. at least 65%, 70%, 75%, 80%, 85%, 95%, 96%, 97%, 98%, 99% or 100%) of SEQ ID NO:3. For example, the bacterial strain for use in the invention may have a chromosome with at least 90% sequence identity to SEQ ID NO:3 across 70% of SEQ ID NO:3, or at least 90% sequence identity to SEQ ID NO:3 across 80% of SEQ ID NO:3, or at least 90% sequence identity to SEQ ID NO:3 across 90% of SEQ ID NO:3, or at least 90% sequence identity to SEQ ID NO:3 across 100% of SEQ ID NO:3, or at least 95% sequence identity to SEQ ID NO:3 across 70% of SEQ ID NO:3, or at least 95% sequence identity to SEQ ID NO:3 across 80% of SEQ ID NO:3, or at least 95% sequence identity to SEQ ID NO:3 across 90% of SEQ ID NO:3, or at least 95% sequence identity to SEQ ID NO:3 across 100% of SEQ ID NO:3, or at least 98% sequence identity to SEQ ID NO:3 across 70% of SEQ ID NO:3, or at least 98% sequence identity to SEQ ID NO:3 across 80% of SEQ ID NO:3, or at least 98% sequence identity to SEQ ID NO:3 across 90% of SEQ ID NO:3, or at least 98% identity to SEQ ID NO:3 across 95% of SEQ ID NO:3, or at least 98% sequence identity to SEQ ID NO:3 across 100% of SEQ ID NO:3, or at least 99.5% sequence identity to SEQ ID NO:3 across 90% of SEQ ID NO:3, or at least 99.5% identity to SEQ ID NO:3 across 95% of SEQ ID NO:3, or at least 99.5% identity to SEQ ID NO:3 across 98% of SEQ ID NO:3, or at least 99.5% sequence identity to SEQ ID NO:3 across 100% of SEQ ID NO:3.

The bacterial strain for use in the invention can be a plasmid with sequence identity to SEQ ID NO:4. The bacterial strain for use in the invention can have a plasmid with at least 90% sequence identity (e.g. at least 92%, 94%, 95%, 96%, 97%, 98%, 99% or 100% sequence identity) to SEQ ID NO:4 across at least 60% (e.g. at least 65%, 70%, 75%, 80%, 85%, 95%, 96%, 97%, 98%, 99% or 100%) of SEQ ID NO:4. For example, the bacterial strain for use in the invention may have a plasmid with at least 90% sequence identity to SEQ ID NO:4 across 70% of SEQ ID NO:4, or at least 90% sequence identity to SEQ ID NO:4 across 80% of SEQ ID NO:4, or at least 90% sequence identity to SEQ ID NO:4 across 90% of SEQ ID NO:4, or at least 90% sequence identity to SEQ ID NO:4 across 100% of SEQ ID NO:4, or at least 95% sequence identity to SEQ ID NO:4 across 70% of SEQ ID NO:4, or at least 95% sequence identity to SEQ ID NO:4 across 80% of SEQ ID NO:4, or at least 95% sequence identity to SEQ ID NO:4 across 90% of SEQ ID NO:4, or at least 95% sequence identity to SEQ ID NO:4 across 100% of SEQ ID NO:4, or at least 98% sequence identity to SEQ ID NO:4 across 70% of SEQ ID NO:4, or at least 98% sequence identity to SEQ ID NO:4 across 80% of SEQ ID NO:4, or at least 98% sequence identity to SEQ ID NO:4 across 90% of SEQ ID NO:4, or at least 98% sequence identity to SEQ ID NO:4 across 100% of SEQ ID NO:4.

The bacterial strain for use in the invention may have a chromosome with sequence identity to SEQ ID NO:3, for example as described above, and a 16S rRNA sequence with sequence identity to any of SEQ ID NO:1 or 2, for example as described above, preferably with a 16S rRNA sequence that is at least 99% identical to SEQ ID NO: 2, more preferably which comprises the 16S rRNA sequence of SEQ ID NO:2, and optionally comprises a plasmid with sequence identity to SEQ ID NO:4, as described above.

The bacterial strain for use in the invention may have a chromosome with sequence identity to SEQ ID NO:3, for example as described above, and optionally comprise a plasmid with sequence identity to SEQ ID NO:4, as described above, and is effective for increasing the microbiota diversity in a subject.

The bacterial strain for use in the invention can have a chromosome with sequence identity to SEQ ID NO:3, for example as described above, and a 16S rRNA sequence with sequence identity to any of SEQ ID NOs: 1 or 2, for example as described above, and optionally comprises a plasmid with sequence identity to SEQ ID NO:4, as described above, and is effective increasing the microbiota diversity in a subject.

The bacterial strain for use in the invention may have a 16S rRNA sequence that is at least 99%, 99.5% or 99.9% identical to the 16S rRNA sequence represented by SEQ ID NO: 2 (for example, which comprises the 16S rRNA sequence of SEQ ID NO:2) and a chromosome with at least 95% sequence identity to SEQ ID NO:3 across at least 90% of SEQ ID NO:3, and optionally comprises a plasmid with sequence identity to SEQ ID NO:4, as described above, and which is effective for increasing the microbiota diversity in a subject.

The bacterial strain for use in the invention can have a 16S rRNA sequence that is at least 99%, 99.5% or 99.9% identical to the 16S rRNA sequence represented by SEQ ID NO: 2 (for example, which comprises the 16S rRNA sequence of SEQ ID NO:2) and a chromosome with at least 98% sequence identity (e.g. at least 99% or at least 99.5% sequence identity) to SEQ ID NO:3 across at least 98% (e.g. across at least 99% or at least 99.5%) of SEQ ID NO:3, and optionally comprises a plasmid with sequence identity to SEQ ID NO:4, as described above, and which is effective for increasing the microbiota diversity in a subject.

The bacterial strain for use in the invention may be Enterococcus gallinarum and may have a 16S rRNA sequence that is at least 99%, 99.5% or 99.9% identical to the 16S rRNA sequence represented by SEQ ID NO: 2 (for example, which comprises the 16S rRNA sequence of SEQ ID NO:2) and a chromosome with at least 98% sequence identity (e.g. at least 99% or at least 99.5% sequence identity) to SEQ ID NO:3 across at least 98% (e.g. across at least 99% or at least 99.5%) of SEQ ID NO:3, and optionally comprises a plasmid with sequence identity to SEQ ID NO:4, as described above, and which is effective for increasing the microbiota diversity in a subject.

References to a percentage sequence identity between two nucleotide sequences refers to the percentage of nucleotides that are the same in comparing the two sequences when aligned. This alignment and percent homology or sequence identity can be determined using software programs known in the art, for example those described in section 7.7.18 of ref. [31]. A preferred alignment is determined by the Smith-Waterman homology search algorithm using an affine gap search with a gap open penalty of 12 and a gap extension penalty of 2, BLOSUM matrix of 62. The Smith-Waterman homology search algorithm is disclosed in ref [32].

Other possible computer programs are BLAST or FASTA, in which two sequences are aligned for optimal matching of their respective residues (either along the full length of one or both sequences or along a pre-determined portion of one or both sequences). The programs provide a default opening penalty and a default gap penalty, and a scoring matrix such as PAM 250 [33] can be used in conjunction with the computer program. For example, the percent identity can then be calculated as: the total number of identical matches multiplied by 100 and then divided by the sum of the length of the longer sequence within the matched span and the number of gaps introduced into the shorter sequences in order to align the two sequences.

Alternatively, strains that are biotypes of the bacterium deposited under accession number NCIMB 42488 or NCIMB 42761 and that are suitable for use in the invention may be identified by using the accession number NCIMB 42488 or NCIMB 42761 deposit and restriction fragment analysis and/or PCR analysis, for example by using fluorescent amplified fragment length polymorphism (FAFLP) and repetitive DNA element (rep)-PCR fingerprinting, or protein profiling, or partial 16S or 23s rDNA sequencing. These techniques may be used to identify other Enterococcus gallinarum or Enterococcus caselliflavus strains.

Strains that are biotypes of the bacterium deposited under accession number NCIMB 42488 or NCIMB 42761 and that are suitable for use in the invention may be strains that provide the same pattern as the bacterium deposited under accession number NCIMB 42488 or NCIMB 42761 when analysed by amplified ribosomal DNA restriction analysis (ARDRA), for example when using Sau3AI restriction enzyme (for exemplary methods and guidance see, for example reference 34). Alternatively, biotype strains may be identified as strains that have the same carbohydrate fermentation patterns as the bacterium deposited under accession number NCIMB 42488 or NCIMB 42761. The carbohydrate fermentation pattern can be determined using the API 50 CHL panel (bioMérieux). The bacterial strain used in the invention can be:
(i) positive for fermentation of at least one of (e.g. at least 2, 3, 4, 5, 6, 7, 8, 9, 10, 11, 12, 13, 14, 15, 16, 17 or all of): L-arabinose, D-ribose, D-xylose, D-galactose, D-glucose, D-fructose, D-mannose, N-acetylglucosamine, amygdalin, arbutin, salicin, D-cellobiose, D-maltose, D-trehalose, gentiobiose, and D-tagatose; and/or
(ii) intermediate for fermentation of at least one of (e.g. at least 2, 3, 4 or all of): D-mannitol, Methyl-αD-glycopyranoside, D-lactose, starch, and;
   preferably as determined by API 50 CHL analysis (preferably using the API 50 CHL panel from bioMérieux).

Preferably biotype strains that are suitable for use in the invention are strains that have a carbohydrate fermentation pattern of:
(i) positive for fermentation of L-arabinose, D-ribose, D-xylose, D-galactose, D-glucose, D-fructose, D-mannose, N-acetylglucosamine, amygdalin, arbutin, esculin, salicin, D-cellobiose, D-maltose, D-trehalose, gentiobiose, and D-tagatose; and/or
(ii) intermediate for fermentation of D-mannitol, Methyl-αD-glycopyranoside, starch, and;
   preferably as determined by API 50 CHL analysis (preferably using the API 50 CHL panel from bioMérieux).

Preferably biotype strains that are suitable for use in the invention are strains that have a carbohydrate fermentation pattern of:
(i) positive for fermentation of L-arabinose, D-xylose, D-galactose, D-glucose, D-fructose, D-mannose, N-acetylglucosamine, amygdalin, arbutin, esculin, salicin, D-cellobiose, D-maltose, D-lactose, D-saccharose (sucrose), D-trehalose and gentiobiose; and/or
(ii) intermediate for fermentation of methyl-αD-glycopyranoside and melibiose
   preferably as determined by API 50 CHL analysis (preferably using the API 50 CHL panel from bioMérieux). Other strains that are useful in the compositions and methods of the invention, such as biotypes of the bacterium deposited under accession number NCIMB 42488 or NCIMB 42761, may be identified using any appropriate method or strategy, including the assays described in the examples. For instance, strains for use in the invention may be identified by culturing in anaerobic YCFA and/or administering the bacteria to the type II collagen-induced arthritis mouse model and then assessing cytokine levels. In particular, bacterial strains that have similar growth patterns, metabolic type and/or surface antigens to the bacterium deposited under accession number NCIMB 42488 or NCIMB 42761 may be useful in the invention. A useful strain will have comparable immune modulatory activity to the NCIMB 42488 strain.

The bacterial strain used in the invention can be:
(i) Positive for at least one of (e.g. at least 2, 3, 4, 5, 6, 7 or all of): mannose fermentation, glutamic acid decarboxylase, arginine arylamidase, phenylalanine arylamidase, pyroglutamic acid arylamidase, tyrosine arylamidase, histidine arylamidase and serine arylamidase; and/or
(ii) Intermediate for at least one of (e.g. at least 2 or all of): β-galactosidase-6-phosphate, β-glucosidase and N-acetyl-β-glucosaminidase; and/or
(iii) Negative for at least one of (e.g. at least 2, 3, 4, 5, 6 or all of): Raffinose fermentation, Proline arylamidase, Leucyl glycine arylamidase, Leucine arylamidase, Alanine arylamidase, Glycine arylamidase and Glutamyl glutamic acid arylamidase, preferably as determined by an assay of carbohydrate, amino acid and nitrate metabolism, and optionally an assay of alkaline phosphatase activity, more preferably as determined by Rapid ID 32A analysis (preferably using the Rapid ID 32A system from bioMérieux).

The bacterial strain used in the invention can be:
(i) Negative for at least one of (e.g. at least 2, 3, or all 4 of) glycine arylamidase, raffinose fermentation, proline arylamidase, and leucine arylamidase, for example, as determined by an assay of carbohydrate, amino acid and nitrate metabolism, preferably as determined by Rapid ID 32A analysis (preferably using the Rapid ID 32A system from bioMérieux); and/or
(ii) Intermediate positive for fermentation of L-fucose, preferably as determined by API 50 CHL analysis (preferably using the API 50 CHL panel from bioMérieux).

The bacterial strain used in the invention is an extracellular ATP producer, for example one which produces 6-6.7 ng/μl (for example, 6.1-6.6 ng/μl or 6.2-6.5 ng/μl or 6.33±0.10 ng/μl) of ATP as measured using the ATP Assay Kit (Sigma-Aldrich, MAK190). Bacterial extracellular ATP can have pleiotropic effects including activation of T cell-receptor mediated signalling [35], promotion of intestinal Th17 cell differentiation [36] and induction of secretion of the pro-inflammatory mediator IL-1β by activating the NLRP3 inflammasome [37]. Accordingly, a bacterial strain which is an extracellular ATP producer is useful for increasing and/or stabilising the microbiota diversity in a subject.

The bacterial strain for use in the invention can comprises one or more of the following three genes: Mobile element protein; Xylose ABC transporter, permease component; and FIG. 00632333: hypothetical protein. For example, the bacterial strain for use in the invention comprises genes encoding Mobile element protein and Xylose ABC transporter, permease component; Mobile element protein and FIG. 00632333: hypothetical protein; Xylose ABC transporter, permease component and FIG. 00632333: hypothetical protein; or Mobile element protein, Xylose ABC transporter, permease component, and FIG. 00632333: hypothetical protein.

As discussed above, the Enterococcus gallinarum or Enterococcus caselliflavus strain for use in the invention may be a strain which has the same safety and therapeutic efficacy characteristics as the strains deposited under accession number NCIMB 42488 or NCIMB 42761. The composition can therefore comprise an Enterococcus gallinarum strain that is not the strain deposited under accession number NCIMB 42488 or NCIMB 42761 but has the same safety and therapeutic efficacy characteristics as the strains deposited under accession number NCIMB 42488 or NCIMB 42761. The safety characteristics of a strain can be established for example by testing the resistance of the strain to antibiotics, for example distinguishing between intrinsic and transmissible resistance to antibiotics. The safety characteristics of a strain can also be established by evaluating the pathogenic properties of a strain in vitro, for example the levels of toxin production. Other safety tests include testing the acute or chronic toxicity of the bacterial strain in rat and mice models. The therapeutic efficacy of a strain can be established by functional characterization of the bacterial strain in vitro and in vivo using a relevant model.

In preferred embodiments, the bacterial strains in the compositions of the invention are viable and capable of partially or totally colonising the intestine.

### Therapeutic Uses

The compositions of the invention are for use in a method of increasing and/or stabilising the microbiota diversity in a subject diagnosed with a disease. The examples demonstrate that administration of the compositions of the invention can lead to increased microbiota diversity. They further show that the compositions of the invention can increase the stability of the microbiota diversity in a subject.

Accordingly, the disease to be treated or prevented using a composition of the invention is preferably a disease associated with a level of microbiota diversity that is reduced relative to the microbiota diversity of a healthy subject and/or a disease that is associated with reduced stability of the microbiota.

The compositions are for use in a subject that exhibits, or is expected to exhibit, reduced levels of microbiota diversity, for instance, when compared to a healthy subject, or a population of healthy subjects. For instance the composition can be for use in treating a subject having less than 101 different bacterial species (e.g. less than 100, 99, 98, 97, 96, 95, 93, 90, 85, 80, 75 or 70 bacterial species) and/or less than 195 different strains (e.g. less than 193, 190, 187, 185, 183, 180, 175, 170, 165, 160, 150, 140 bacterial strains) in its microbiota. For example, the composition can be for use in treating a subject that has at least one bacterial genus (e.g. at least 2, 3, 4, 5, 6, 7, 8, 9 or 10 bacterial genera) fewer in its intestinal microbiota compared to a healthy subject or compared to a population of healthy subjects. The treatment or prevention can comprise a step of diagnosing a subject as having a reduced level of microbiota diversity and then if a reduced level of diversity is found to be present, the subject is then treated with a composition according to the invention.

Reduced diversity of the microbiota is associated with numerous pathological diseases, and the examples demonstrate that the compositions of the invention may be effective at increasing and/or stabilising the microbiota diversity in a subject. A skilled person can easily identify suitable diseases which would benefit from the increase in microbiota diversity/stability effected by the compositions of the invention by assessing the microbiota diversity and/or stability in a patient and comparing it with those of a healthy subject.

The pathogenesis of the disease can affect the intestine. However, in other cases the pathogenesis of the disease does not affect the intestine or is not localised at the intestine. The treatment or prevention of the disease can occur in the intestine or it can occur at a distal site. The disease that is being treated may be systemic.

Examples of diseases characterised by reduced diversity of the microbiota which may be treated using the compositions of the present invention include neurological conditions such as Alzheimer's disease, Parkinson's disease, autism and multiple sclerosis; gastrointestinal disorders such as irritable bowel syndrome and inflammatory bowel disease; musculoskeletal disorders such as rheumatoid arthritis and psoriatic arthritis; metabolic disorders including Type I diabetes; and wasting/fatigue conditions including sarcopenia and myalgic encephalomyelitis.

The examples show that treatment with compositions of the invention leads to increased microbiota diversity and/or stability of the microbiota. Therefore, the compositions as described above are useful in a method of increasing and/or stabilising the microbiota diversity in a subject. As explained above, there are a substantial number of diseases associated with reductions in microbiome diversity. A reduction in microbiome diversity may be caused and/or exacerbated by certain diseases (e.g. cancer) or the therapies used to treat those diseases.

The compositions of the invention can be used for increasing and/or stabilising the microbiota diversity in a subject diagnosed with cancer. Links have previously been suggested between the gut microbiome and a number of cancers. For example, taxonomic analysis of faecal samples from patients with colorectal cancer showed a decrease in the diversity of the microbiota compared to healthy patients. Increasing the diversity of the microbiota may therefore not only prevent or treat diseases characterised by a reduction in microbiome diversity, but advantageously also contribute to the prevention or treatment of cancer such as colorectal cancer.

The compositions are for use in subjects that exhibit, or are expected to exhibit, reduced stability of the microbiota diversity, for instance, when compared to a healthy subject, or a population of healthy subjects. The treatment or prevention can comprise a step of diagnosing a subject as having a reduced stability in its microbiota and then if reduced stability is found to be present, the subject is then treated with a composition according to the invention.

A composition comprising a bacterial strain of the genus Enterococcus, as defined herein, may also be used in combination with cyclophosphamide for use in treating cancer, an inflammatory disorder or an autoimmune disease.

Bacteria in the microbiota may be detected in faeces from a subject, using standard techniques, such as the qPCR techniques used in the examples.

The subject can be an infant (a subject between the ages 0-1 years), a child (a subject between the ages of 1-18 years) or an adult (a subject with an age above 18 years). The subject can be a healthy subject, in which the composition can be used to prevent a disease, optionally the healthy subject may be one identified as being at risk of developing a disease characterised by a reduction in microbiota diversity.

The subject can have previously received, is receiving, or will be receiving antibiotic treatment (e.g. within one week or month from administration of a composition according to the invention). The treatment can comprises administering the composition of the invention after, together with, or before antibiotic treatment. The composition of the invention and the one or more antibiotics may be for separate, simultaneous or sequential administration.

The composition of the invention can be for use in a method of increasing and/or stabilising the microbiota diversity in a subject having an increased level of hydrogen in their breath relative to a healthy subject. The composition of the invention can be for use in reducing the hydrogen level in the breath of a subject exhibiting or who is expected to exhibit a reduced level of diversity and/or the stability of the of its microbiota. The subject may be a subject diagnosed as having cancer. Treatment with a composition of the invention reduces the level of hydrogen detected in hydrogen breath tests. Accordingly, the hydrogen levels are preferably assessed using a hydrogen breath test. The hydrogen breath test is well known in the art and so the skilled person will know how to conduct such a test and can involve administering lactulose to the subject.

The hydrogen breath test is also a useful tool for monitoring the effectiveness or likely effectiveness of increasing or stabilising the microbiota diversity after treatment using a composition or a combination therapy of the invention. For example, a reduction in the level of hydrogen detected in a subject's breath following treatment with a composition or combination therapy of the invention may indicate that the treatment is increasing the microbiota diversity in the subject. Accordingly, the methods and uses of the invention further comprise monitoring the hydrogen level in a subject's breath during and/or following treatment with a composition of the invention and thereby assessing the effectiveness or likely effectiveness of increasing the microbiota diversity in the subject. For example, hydrogen levels may be monitored at one or more (e.g. 1, 2, 3, 4 or more than 4) times, including before treatment, at the start of treatment, during treatment, at the end of treatment and/or following treatment, as desired. The level of hydrogen in the subject's breath at the end and/or following the dosing period (during which the composition is administered to the subject) is compared to the level at the start and/or before the dosing period and a reduction in the level indicates the effectiveness or likely effectiveness of increasing the microbiota diversity in the subject. The hydrogen level in the subject's breath can be measured at multiple times. For example, if the dosing period is 16 days, it may be desirable to take measurements at day 1 and day 16, or for example at day 1, day 2, day 15 and day 16. Multiple measurements can be taken and the mean of those measurements obtained (for example, the mean of day 1 and day 2 and the mean of day 15 and day 16). A reduction in at least 40 ppm in the hydrogen level Cmax indicates that the composition is effective or likely to be effective at increasing the microbiota diversity in the subject. The hydrogen breath test is a standard assay and so predetermined levels are known in the art.

The inventors have shown that the abundance of Barnesiella intestinihominis was increased following administration of a composition comprising a bacterial strain of the genus Enterococcus, as defined herein, as evidenced by the examples of the present specification. That organism has been shown to have an immunostimulatory effect. More specifically, that organism has been shown to promote cyclophosphamide-induced therapeutic immunomodulatory effects through the infiltration of IFN-producing T cells in cancer lesions. Furthermore, Barnesiella intestinihominis specific-memory Th1 cell immune responses selectively predicted longer progression-free survival in advanced lung and ovarian cancer patients treated with chemo-immunotherapy. [38]. By using a composition comprising a bacterial strain of the genus Enterococcus, the invention thus increases the levels of Barnesiella intestinihominis and so promotes cyclophosphamide efficacy. The combination therapy of the invention is therefore particularly useful for treating diseases which are known to benefit from cyclophosphamide treatment as the combination will enhance the efficacy of the treatment. Known diseases which can be treated with cyclophosphamide comprise cancer, inflammatory and autoimmune disorders.

Through the administration of the compositions of the invention, not only is microbiome diversity stabilised (if not increased) but advantageously, the abundance of Barnesiella intestinihominis is also increased. Thus, the compositions of the invention are particularly useful in stabilising or improving the microbiome diversity in cancer patients.

The compositions of the invention may therefore increase the levels of Barnesiella intestinihominis species. A skilled person will understand that this increase will be relative to the levels of the Barnesiella intestinihominis species prior to administration of the composition.

Barnesiella intestinihominis has been reported to enhance the immunomodulatory effect of cyclophosphamide [38]. The data in the examples therefore confirm that the combination of a composition comprising a bacterial strain of the species Enterococcus gallinarum with cyclophosphamide is particularly useful. Thus, according to an aspect of the present invention, there is provided the use in therapy of a combination of a composition comprising a bacterial strain of the genus Enterococcus and cyclophosphamide. Preferably the bacterial strain is of the species Enterococcus gallinarum.

In such embodiments, the composition comprising the bacterial strain of the genus Enterococcus may be administered to increase levels of Barnesiellla intestinihominis in the gastrointestinal tract of a patient to enhance the immunomodulatory effect of cyclophosphamide.

In embodiments of this aspect of the invention, the combination of a composition comprising a bacterial strain of the genus Enterococcus and cyclophosphamide may be used to treat cancer. For example, the combination may be for use in reducing tumour size, reducing tumour growth, or reducing angiogenesis in the treatment of cancer.

In certain embodiments, the combination is for use in treating or preventing colorectal cancer, such as colon cancer, preferably colorectal adenocarcinoma. In some embodiments, the cancer is of the intestine. In other embodiments, the combination is for use in treating or preventing lung cancer, lymphoma, multiple myeloma, leukemia, ovarian cancer, breast cancer (in particular carcinoma), small cell lung cancer, neuroblastoma, sarcoma, retinoblastoma, adenocarcinoma (in particular of the ovary) or liver cancer. In other embodiments, the compositions of the invention are for use in treating or preventing carcinoma.

In embodiments, the combination of a composition comprising a bacterial strain of the genus Enterococcus and cyclophosphamide is useful for treating an autoimmune or inflammatory disease. Examples of diseases (which are known to respond to treatment with cyclophosphamide) which may be treated with the combination include nephrotic syndrome, systemic lupus erythematosus, granulomatosis with polyangiitis, aplastic anemia, microscopic polyangiitis, polyarteritis nodosa, eosinophilic granulomatosis with polyangiitis (Churg-Strauss syndrome), Behçet syndrome, primary angiitis of the central nervous system, isolated vasculitic neuropathy, following organ transplant and in preparation for bone marrow transplantation. Generally, the combination of a composition of the invention and cyclophosphamide will be used for a disease where an increase and/or stabilisation of the microbiota diversity in the subject suffering from the disease is expected to be beneficial.

In addition to their positive effects on levels of Barnesiella intestihominis (and improving microbiome diversity) the compositions have also advantageously been demonstrated in the examples to increase the levels of a number of short-chain fatty acid producing bacteria, including those from the genera Lachnospiraceae and Roseburia. Organisms belonging to those genera have been associated with the treatment of several diseases including inflammatory bowel disease (in the case of organisms belonging to the genus Roseburia [39]) and weight loss (in the case of members of the Lachnospiraceae genus).

The compositions of the invention may therefore increase the levels of Lachnospiraceae and/or Roseburia species. A skilled person will understand that this increase will be relative to the levels of the Lachnospiraceae and/or Roseburia species (respectively) prior to administration of the composition.

The examples demonstrate that the compositions can increase the abundance of bacteria from the genera Lachnospiraceae. A reduction in the levels of bacteria from the genera Lachnospiraceae in the human microbiota have been linked to gastrointestinal diseases such as inflammatory bowel disease (IBD), ulcerative colitis and Crohn's disease [40]. Cyclophosphamide treatment has been shown to reduce the level of numerous bacterial species including those from the genera Lachnospiraceae [41].

Therefore, in some embodiments the combination of a composition comprising a bacterial strain of the genus Enterococcus and cyclophosphamide can used to increase levels of the genera Lachnospiraceae in the gastrointestinal tract. Administering cyclophosphamide in combination with a bacterial strain of the genus Enterococcus prevents a decrease in the relevance of bacteria genera Lachnospiraceae that is associated with cyclophosphamide treatment. Increasing the levels of the genera Lachnospiraceae may prevent or treat gastrointestinal diseases such as inflammatory bowel disease, ulcerative colitis and Crohn's disease.

The compositions of the invention have further been shown to influence the levels of Clostridium species. These organisms have been associated with modulation of the immune system and efficacy in the prevention and treatment of autoimmune, infectious and allergic diseases.

The examples have also shown that the compositions of the invention can increase the levels of bacteria from the genera Alistipes. Patients suffering from IBD have a decrease in their microbiota diversity. Taxa that are significantly depleted compared to healthy controls include bacteria from the genera Alistipes and Barnesiella [42]. Therefore, the compositions of the invention may be useful in treating or preventing gastrointestinal diseases such as inflammatory bowel disease, ulcerative colitis and Crohn's disease. The compositions of the invention may treat gastrointestinal diseases such as inflammatory bowel disease, ulcerative colitis and Crohn's disease by increasing the levels of include bacteria from the genera Alistipes and/or Barnesiella.

Compositions of the invention have been demonstrated in the examples to exert a limiting effect on the pentose phosphate pathway, as characterised by a reduction in the formation of metabolites including ribose 5-phosphate, erythrose 4-phosphate and sedoheptulose 7-phosphate.

Again, this property of the compositions of the present invention is advantageous because this pathway has been implicated as having a protective effect in assisting glycolytic cancer cells to tackle oxidative stress, and could therefore be protective to tumour cells. Literature has further shown that Glucose-6-phosphate dehydrogenase deficiency can protect against cancer, in particular breast, colorectal cancer etc. and that an increase in members of the pentose phosphate pathway is associated with poor outcomes in cancer patients [43,44]. Thus, when the compositions of the present invention are used to promote the diversity of the microbiome in subjects that would benefit from a reduction in the pentose phosphate pathway (e.g. cancer patients), they may additionally exert this effect.

Thus, according to a further aspect of the present invention, there is provided a composition comprising a bacterial strain of the genus Enterococcus, for use in reducing the formation in vivo of at least one metabolite associated with the pentose phosphate pathway in a subject in need of such treatment, for example, at risk of or diagnosed with a disease caused or exacerbated by normal or elevated function of the pentose phosphate. For example, the metabolite may be ribose 5-phosphate, erythrose 4-phosphate and sedoheptulose 7-phosphate.

The compositions of the invention can be used to improve and/or stabilise the microbiota diversity in a patient that has previously received chemotherapy. The compositions of the invention may be used to improve and/or stabilise the microbiota diversity in a patient that has not tolerated a chemotherapy treatment.

The compositions of the invention may also be useful in increasing and/or stabilising microbiota diversity in a patient diagnosed with acute lymphoblastic leukemia (ALL), acute myeloid leukemia, adrenocortical carcinoma, basal-cell carcinoma, bile duct cancer, bladder cancer, bone tumor, osteosarcoma/malignant fibrous histiocytoma, brainstem glioma, brain tumor, cerebellar astrocytoma, cerebral astrocytoma/malignant glioma, ependymoma, medulloblastoma, supratentorial primitive neuroectodermal tumors, breast cancer, bronchial adenomas/carcinoids, Burkitt's lymphoma, carcinoid tumor, cervical cancer, chronic lymphocytic leukemia, chronic myelogenous leukemia, chronic myeloproliferative disorders, colon cancer, cutaneous T-cell lymphoma, endometrial cancer, ependymoma, esophageal cancer, Ewing's sarcoma, intraocular melanoma, retinoblastoma, gallbladder cancer, gastric cancer, gastrointestinal carcinoid tumor, gastrointestinal stromal tumor (GIST), germ cell tumor, glioma, childhood visual pathway and hypothalamic, Hodgkin lymphoma, melanoma, islet cell carcinoma, Kaposi sarcoma, renal cell cancer, laryngeal cancer, leukaemias, lymphomas, mesothelioma, neuroblastoma, non-Hodgkin lymphoma, oropharyngeal cancer, osteosarcoma, ovarian cancer, pancreatic cancer, parathyroid cancer, pharyngeal cancer, pituitary adenoma, plasma cell neoplasia, prostate cancer, renal cell carcinoma, retinoblastoma, sarcoma, testicular cancer, thyroid cancer, or uterine cancer.

The compositions of the invention may be used to increase and/or stabilise microbiota diversity in a patient being treated with further therapeutic agents. The compositions of the invention may be combined with anti-cancer agents. Preferably, the invention provides a composition comprising a bacterial strain of the species Enterococcus gallinarum and an anticancer agent. Preferably, the anticancer agent is an immune checkpoint inhibitor, a targeted antibody immunotherapy, a CAR-T cell therapy, an oncolytic virus, or a cytostatic drug. In preferred embodiments, the composition comprises an anti-cancer agent selected from the group consisting of: Yervoy (ipilimumab, BMS); Keytruda (pembrolizumab, Merck); Opdivo (nivolumab, BMS); MEDI4736 (AZ/MedImmune); MPDL3280A (Roche/Genentech); Tremelimumab (AZ/MedImmune); CT-011 (pidilizumab, CureTech); BMS-986015 (lirilumab, BMS); MEDI0680 (AZ/MedImmune); MSB-0010718C (Merck); PF-05082566 (Pfizer); MEDI6469 (AZ/MedImmune); BMS-986016 (BMS); BMS-663513 (urelumab, BMS); IMP321 (Prima Biomed); LAG525 (Novartis); ARGX-110 (arGEN-X); PF-05082466 (Pfizer); CDX-1127 (varlilumab; CellDex Therapeutics); TRX-518 (GITR Inc.); MK-4166 (Merck); JTX-2011 (Jounce Therapeutics); ARGX-115 (arGEN-X); NLG-9189 (indoximod, NewLink Genetics); INCB024360 (Incyte); IPH2201 (Innate Immotherapeutics/AZ); NLG-919 (NewLink Genetics); anti-VISTA (JnJ); Epacadostat (INCB24360, Incyte); F001287 (Flexus/BMS); CP 870893 (University of Pennsylvania); MGA271 (Macrogenix); Emactuzumab (Roche/Genentech); Galunisertib (Eli Lilly); Ulocuplumab (BMS); BKT140/BL8040 (Biokine Therapeutics); Bavituximab (Peregrine Pharmaceuticals); CC 90002 (Celgene); 852A (Pfizer); VTX-2337 (VentiRx Pharmaceuticals); IMO-2055 (Hybridon, Idera Pharmaceuticals); LY2157299 (Eli Lilly); EW-7197 (Ewha Women's University, Korea); Vemurafenib (Plexxikon); Dabrafenib (Genentech/GSK); BMS-777607 (BMS); BLZ945 (Memorial Sloan-Kettering Cancer Centre); Unituxin (dinutuximab, United Therapeutics Corporation); Blincyto (blinatumomab, Amgen); Cyramza (ramucirumab, Eli Lilly); Gazyva (obinutuzumab, Roche/Biogen); Kadcyla (ado-trastuzumab emtansine, Roche/Genentech); Perj eta (pertuzumab, Roche/Genentech); Adcetris (brentuximab vedotin, Takeda/Millennium); Arzerra (ofatumumab, GSK); Vectibix (panitumumab, Amgen); Avastin (bevacizumab, Roche/Genentech); Erbitux (cetuximab, BMS/Merck); Bexxar (tositumomab-I131, GSK); Zevalin (ibritumomab tiuxetan, Biogen); Campath (alemtuzumab, Bayer); Mylotarg (gemtuzumab ozogamicin, Pfizer); Herceptin (trastuzumab, Roche/Genentech); Rituxan (rituximab, Genentech/Biogen); volociximab (Abbvie); Enavatuzumab (Abbvie); ABT-414 (Abbvie); Elotuzumab (Abbvie/BMS); ALX-0141 (Ablynx); Ozaralizumab (Ablynx); Actimab-C(Actinium); Actimab-P (Actinium); Milatuzumab-dox (Actinium); Emab-SN-38 (Actinium); Naptumonmab estafenatox (Active Biotech); AFM13 (Affimed); AFM11 (Affimed); AGS-16C3F (Agensys); AGS-16M8F (Agensys); AGS-22ME (Agensys); AGS-15ME (Agensys); GS-67E (Agensys); ALXN6000 (samalizumab, Alexion); ALT-836 (Altor Bioscience); ALT-801 (Altor Bioscience); ALT-803 (Altor Bioscience); AMG780 (Amgen); AMG228 (Amgen); AMG820 (Amgen); AMG172 (Amgen); AMG595 (Amgen); AMG110 (Amgen); AMG232 (adecatumumab, Amgen); AMG211 (Amgen/MedImmune); BAY20-10112 (Amgen/Bayer); Rilotumumab (Amgen); Denosumab (Amgen); AMP-514 (Amgen); MEDI575 (AZ/MedImmune); MEDI3617 (AZ/MedImmune); MEDI6383 (AZ/MedImmune); MEDI551 (AZ/MedImmune); Moxetumomab pasudotox (AZ/MedImmune); MEDI565 (AZ/MedImmune); MEDI0639 (AZ/MedImmune); MEDI0680 (AZ/MedImmune); MEDI562 (AZ/MedImmune); AV-380 (AVEO); AV203 (AVEO); AV299 (AVEO); BAY79-4620 (Bayer); Anetumab ravtansine (Bayer); vantictumab (Bayer); BAY94-9343 (Bayer); Sibrotuzumab (Boehringer Ingleheim); BI-836845 (Boehringer Ingleheim); B-701 (BioClin); BIIB015 (Biogen); Obinutuzumab (Biogen/Genentech); BI-505 (Bioinvent); BI-1206 (Bioinvent); TB-403 (Bioinvent); BT-062 (Biotest) BIL-010t (Biosceptre); MDX-1203 (BMS); MDX-1204 (BMS); Necitumumab (BMS); CAN-4 (Cantargia AB); CDX-011 (Celldex); CDX1401 (Celldex); CDX301 (Celldex); U3-1565 (Daiichi Sankyo); patritumab (Daiichi Sankyo); tigatuzumab (Daiichi Sankyo); nimotuzumab (Daiichi Sankyo); DS-8895 (Daiichi Sankyo); DS-8873 (Daiichi Sankyo); DS-5573 (Daiichi Sankyo); MORab-004 (Eisai); MORab-009 (Eisai); MORab-003 (Eisai); MORab-066 (Eisai); LY3012207 (Eli Lilly); LY2875358 (Eli Lilly); LY2812176 (Eli Lilly); LY3012217(Eli Lilly); LY2495655 (Eli Lilly); LY3012212 (Eli Lilly); LY3012211 (Eli Lilly); LY3009806 (Eli Lilly); cixutumumab (Eli Lilly); Flanvotumab (Eli Lilly); IMC-TR1 (Eli Lilly); Ramucirumab (Eli Lilly); Tabalumab (Eli Lilly); Zanolimumab (Emergent Biosolution); FG-3019 (FibroGen); FPA008 (Five Prime Therapeutics); FP-1039 (Five Prime Therapeutics); FPA144 (Five Prime Therapeutics); catumaxomab (Fresenius Biotech); IMAB362 (Ganymed); IMAB027 (Ganymed); HuMax-CD74 (Genmab); HuMax-TFADC (Genmab); GS-5745 (Gilead); GS-6624 (Gilead); OMP-21M18 (demcizumab, GSK); mapatumumab (GSK); IMGN289 (ImmunoGen); IMGN901 (ImmunoGen); IMGN853 (ImmunoGen); IMGN529 (Immuno Gen); IMMU-130 (Immunomedics); milatuzumab-dox (Immunomedics); IM MU-115 (Immunomedics); IMMU-132 (Immunomedics); IM MU-106 (Immunomedics); IMMU-102 (Immunomedics); Epratuzumab (Immunomedics); Clivatuzumab (Immunomedics); IPH41 (Innate Immunotherapeutics); Daratumumab (Janssen/Genmab); CNTO-95 (Intetumumab, Janssen); CNTO-328 (siltuximab, Janssen); KB004 (KaloBios); mogamulizumab (Kyowa Hakko Kirrin); KW-2871 (ecromeximab, Life Science); Sonepcizumab (Lpath); Margetuximab (Macrogenics); Enoblituzumab (Macrogenics); MGD006 (Macrogenics); MGF007 (Macrogenics); MK-0646 (dalotuzumab, Merck); MK-3475 (Merck); Sym004 (Symphogen/Merck Serono); DI17E6 (Merck Serono); MOR208 (Morphosys); MOR202 (Morphosys); Xmab5574 (Morphosys); BPC-1C (ensituximab, Precision Biologics); TAS266 (Novartis); LFA102 (Novartis); BHQ880 (Novartis/Morphosys); QGE031 (Novartis); HCD122 (lucatumumab, Novartis); LJM716 (Novartis); AT355 (Novartis); OMP-21M18 (Demcizumab, OncoMed); OMP52M51 (Oncomed/GSK); OMP-59R5 (Oncomed/GSK); vantictumab (Oncomed/Bayer); CMC-544 (inotuzumab ozogamicin, Pfizer); PF-03446962 (Pfizer); PF-04856884 (Pfizer); PSMA-ADC (Progenics); REGN1400 (Regeneron); REGN910 (nesvacumab, Regeneron/Sanofi); REGN421 (enoticumab, Regeneron/Sanofi); RG7221, RG7356, RG7155, RG7444, RG7116, RG7458, RG7598, RG7599, RG7600, RG7636, RG7450, RG7593, RG7596, DCDS3410A, RG7414 (parsatuzumab), RG7160 (imgatuzumab), RG7159 (obintuzumab), RG7686, RG3638 (onartuzumab), RG7597 (Roche/Genentech); SAR307746 (Sanofi); SAR566658 (Sanofi); SAR650984 (Sanofi); SAR153192 (Sanofi); SAR3419 (Sanofi); SAR256212 (Sanofi), SGN-LIV1A (lintuzumab, Seattle Genetics); SGN-CD33A (Seattle Genetics); SGN-75 (vorsetuzumab mafodotin, Seattle Genetics); SGN-19A (Seattle Genetics) SGN-CD70A (Seattle Genetics); SEA-CD40 (Seattle Genetics); ibritumomab tiuxetan (Spectrum); MLN0264 (Takeda); ganitumab (Takeda/Amgen); CEP-37250 (Teva); TB-403 (Thrombogenic); VB4-845 (Viventia); Xmab2512 (Xencor); Xmab5574 (Xencor); nimotuzumab (YM Biosciences); Carlumab (Janssen); NY-ESO TCR (Adaptimmune); MAGE-A-10 TCR (Adaptimmune); CTL019 (Novartis); JCAR015 (Juno Therapeutics); KTE-C19 CAR (Kite Pharma); UCART19 (Cellectis); BPX-401 (Bellicum Pharmaceuticals); BPX-601 (Bellicum Pharmaceuticals); ATTCK20 (Unum Therapeutics); CAR-NKG2D (Celyad); Onyx-015 (Onyx Pharmaceuticals); H101 (Shanghai Sunwaybio); DNX-2401 (DNAtrix); VCN-01 (VCN Biosciences); Colo-Adl (PsiOxus Therapeutics); ProstAtak (Advantagene); Oncos-102 (Oncos Therapeutics); CG0070 (Cold Genesys); Pexa-vac (JX-594, Jennerex Biotherapeutics); GL-ONC1 (Genelux); T-VEC (Amgen); G207 (Medigene); HF10 (Takara Bio); SEPREHVIR (HSV1716, Virttu Biologics); OrienX010 (OrienGene Biotechnology); Reolysin (Oncolytics Biotech); SVV-001 (Neotropix); Cacatak (CVA21, Viralytics); Alimta (Eli Lilly), cisplatin, oxaliplatin, irinotecan, folinic acid, methotrexate, cyclophosphamide, 5-fluorouracil, Zykadia (Novartis), Tafinlar (GSK), Xalkori (Pfizer), Iressa (AZ), Gilotrif (Boehringer Ingelheim), Tarceva (Astellas Pharma), Halaven (Eisai Pharma), Veliparib (Abbvie), AZD9291 (AZ), Alectinib (Chugai), LDK378 (Novartis), Genetespib (Synta Pharma), Tergenpumatucel-L (NewLink Genetics), GV1001 (Kael-GemVax), Tivantinib (ArQule); Cytoxan (BMS); Oncovin (Eli Lilly); Adriamycin (Pfizer); Gemzar (Eli Lilly); Xeloda (Roche); Ixempra (BMS); Abraxane (Celgene); Trelstar (Debiopharm); Taxotere (Sanofi); Nexavar (Bayer); IMMU-132 (Immunomedics); E7449 (Eisai); Thermodox (Celsion); Cometriq (Exellxis); Lonsurf (Taiho Pharmaceuticals); Camptosar (Pfizer); UFT (Taiho Pharmaceuticals); and TS-1 (Taiho Pharmaceuticals).

### Modes of Administration

Preferably, the compositions of the invention are to be administered to the gastrointestinal tract in order to enable delivery to and/or partial or total colonisation of the intestine with the bacterial strain of the invention. Generally, the compositions of the invention are administered orally, but they may be administered rectally, intranasally, or via buccal or sublingual routes.

The compositions of the invention may be administered as a foam, as a spray or a gel. The compositions of the invention may be administered as a suppository, such as a rectal suppository, for example in the form of a theobroma oil (cocoa butter), synthetic hard fat (e.g. suppocire, witepsol), glycero-gelatin, polyethylene glycol, or soap glycerin composition.

The composition of the invention may be administered to the gastrointestinal tract via a tube, such as a nasogastric tube, orogastric tube, gastric tube, jejunostomy tube (J tube), percutaneous endoscopic gastrostomy (PEG), or a port, such as a chest wall port that provides access to the stomach, jejunum and other suitable access ports.

The compositions of the invention may also be formulated for intravenous, rectal, sublingual, subcutaneous or nasal administration.

The compositions of the invention may be administered once, or they may be administered sequentially as part of a treatment regimen. For example, the compositions of the invention can be administered daily.

Sometimes treatment according to the invention can be accompanied by assessment of the patient's gut microbiota. Treatment may be repeated if delivery of and/or partial or total colonisation with the strain of the invention is not achieved such that efficacy is not observed, or treatment may be ceased if delivery and/or partial or total colonisation is successful and efficacy is observed.

The composition of the invention may be administered to a pregnant animal, for example a mammal such as a human in order to promote the increase in microbiota diversity and/or the stability of the microbiota after the child is born.

The compositions of the invention may be administered to a patient that has been identified as having an abnormal gut microbiota. For example, the patient may have reduced or absent colonisation by Enterococcus gallinarum or Enterococcus caselliflavus.

The compositions of the invention may be administered as a food product, such as a nutritional supplement.

Preferably, the compositions of the invention are for the treatment of humans, although they may be used to treat animals including monogastric mammals such as poultry, pigs, cats, dogs, horses or rabbits. The compositions of the invention may be useful for enhancing the growth and performance of animals. If administered to animals, oral gavage may be used.

In embodiments of the invention in which a composition comprising a bacterial strain of the species Enterococcus gallinarum is used in therapy in combination with cyclophosphamide, the cyclophosphamide may be administered as part of the composition or it may be administered separately. Where cyclophosphamide is administered separately, it may be given either concurrently (for example during the same visit to a health care professional) or sequentially. It may also be given via the same route as a composition of the invention or it may be administered differently.

Preferably, cyclophosphamide is administered orally or intravenously (optionally via injection or infusion). Where cyclophosphamide is administered orally, it may be given at a daily dose of 1000 mg or less, 800 mg or less, 500 mg or less, or 200 mg or less. The daily dose may be between 10-500 mg, 50-250 mg or 80-150 mg. Where cyclophosphamide is administered intravenously, it may be given at a daily dose of 500 to 2000 mg.

### Compositions

Generally, the composition of the invention comprises bacteria. The composition can be formulated in freeze-dried form. For example, the composition of the invention may comprise granules or gelatin capsules, for example hard gelatin capsules, comprising a bacterial strain as described above.

Alternatively, the composition of the invention may comprise a live, active bacterial culture. The bacterial strain in the composition of the invention has therefore not been inactivated, killed and/or attenuated, for example by heat. The bacterial strain in the composition of the invention can be viable and/or capable of partially or totally colonising the intestine. The composition can comprises a mixture of live bacterial strains and bacterial strains that have been killed.

The composition of the invention can be encapsulated to enable delivery of the bacterial strain to the intestine. Encapsulation protects the composition from degradation until delivery at the target location through, for example, rupturing with chemical or physical stimuli such as pressure, enzymatic activity, or physical disintegration, which may be triggered by changes in pH. Any appropriate encapsulation method may be used. Exemplary encapsulation techniques include entrapment within a porous matrix, attachment or adsorption on solid carrier surfaces, self-aggregation by flocculation or with cross-linking agents, and mechanical containment behind a microporous membrane or a microcapsule. Guidance on encapsulation that may be useful for preparing compositions of the invention is available in, for example, references [45] and [46].

The composition may be administered orally and may be in the form of a tablet, capsule or powder. Encapsulated products are preferred because Enterococcus gallinarum are anaerobes. Other ingredients (such as vitamin C, for example), may be included as oxygen scavengers and prebiotic substrates to improve the delivery and/or partial or total colonisation and survival in vivo. Alternatively, the probiotic composition of the invention may be administered orally as a food or nutritional product, such as milk or whey based fermented dairy product, or as a pharmaceutical product.

The composition may be formulated as a probiotic.

A composition of the invention includes a therapeutically effective amount of a bacterial strain of the invention. A therapeutically effective amount of a bacterial strain is sufficient to exert a beneficial effect upon a patient. A therapeutically effective amount of a bacterial strain may be sufficient to result in delivery to and/or partial or total colonisation of the patient's intestine. A therapeutically effective amount of a bacterial strain can be established by comparing the ability of the bacterial strain of interest to exert a significant relevant therapeutic effect in an in vitro or in vivo model, as described previously, compared to an untreated control.

A suitable daily dose of the bacteria, for example for an adult human, may be from about 1×10³ to about 1×10¹¹ colony forming units (CFU); for example, from about 1×10⁷ to about 1×10¹⁰ CFU; in another example from about 1×10⁶ to about 1×10¹⁰ CFU. The composition can contain the bacterial strain in an amount of from about 1×10⁶ to about 1×10¹¹ CFU/g, respect to the weight of the composition; for example, from about 1×10⁸ to about 1×10¹⁰ CFU/g. The dose may be, for example, 1 g, 3 g, 5 g, and 10 g.

Typically, a probiotic, such as the composition of the invention, is optionally combined with at least one suitable prebiotic compound. A prebiotic compound is usually a non-digestible carbohydrate such as an oligo- or polysaccharide, or a sugar alcohol, which is not degraded or absorbed in the upper digestive tract. Known prebiotics include commercial products such as inulin and transgalacto-oligosaccharides.

The probiotic composition of the present invention may include a prebiotic compound in an amount of from about 1 to about 30% by weight, respect to the total weight composition, (e.g. from 5 to 20% by weight). Carbohydrates may be selected from the group consisting of: fructo-oligosaccharides (or FOS), short-chain fructo-oligosaccharides, inulin, isomalt-oligosaccharides, pectins, xylo-oligosaccharides (or XOS), chitosan-oligosaccharides (or COS), beta-glucans, arable gum modified and resistant starches, polydextrose, D-tagatose, acacia fibers, carob, oats, and citrus fibers. In one aspect, the prebiotics are the short-chain fructo-oligosaccharides (for simplicity shown herein below as FOSs-c.c); said FOSs-c.c. are not digestible carbohydrates, generally obtained by the conversion of the beet sugar and including a saccharose molecule to which three glucose molecules are bonded.

The compositions of the invention may comprise pharmaceutically acceptable excipients or carriers. Examples of such suitable excipients may be found in reference [47]. Acceptable carriers or diluents for therapeutic use are well known in the pharmaceutical art and are described, for example, in reference [48]. Examples of suitable carriers include lactose, starch, glucose, methyl cellulose, magnesium stearate, mannitol, sorbitol and the like. Examples of suitable diluents include ethanol, glycerol and water. The choice of pharmaceutical carrier, excipient or diluent can be selected with regard to the intended route of administration and standard pharmaceutical practice. The pharmaceutical compositions may comprise as, or in addition to, the carrier, excipient or diluent any suitable binder(s), lubricant(s), suspending agent(s), coating agent(s), solubilising agent(s). Examples of suitable binders include starch, gelatin, natural sugars such as glucose, anhydrous lactose, free-flow lactose, beta-lactose, corn sweeteners, natural and synthetic gums, such as acacia, tragacanth or sodium alginate, carboxymethyl cellulose and polyethylene glycol. Examples of suitable lubricants include sodium oleate, sodium stearate, magnesium stearate, sodium benzoate, sodium acetate, sodium chloride and the like. Preservatives, stabilizers, dyes and even flavouring agents may be provided in the pharmaceutical composition. Examples of preservatives include sodium benzoate, sorbic acid and esters of p-hydroxybenzoic acid. Antioxidants and suspending agents may be also used.

The compositions of the invention may be formulated as a food product. For example, a food product may provide nutritional benefit in addition to the therapeutic effect of the invention, such as in a nutritional supplement. Similarly, a food product may be formulated to enhance the taste of the composition of the invention or to make the composition more attractive to consume by being more similar to a common food item, rather than to a pharmaceutical composition. The composition of the invention is formulated as a milk-based product. The term “milk-based product” means any liquid or semi-solid milk- or whey-based product having a varying fat content. The milk-based product can be, e.g., cow's milk, goat's milk, sheep's milk, skimmed milk, whole milk, milk recombined from powdered milk and whey without any processing, or a processed product, such as yoghurt, curdled milk, curd, sour milk, sour whole milk, butter milk and other sour milk products. Another important group includes milk beverages, such as whey beverages, fermented milks, condensed milks, infant or baby milks; flavoured milks, ice cream; milk-containing food such as sweets.

The compositions of the invention can contain a single bacterial strain or species and may not contain any other bacterial strains or species. Such compositions may comprise only de minimis or biologically irrelevant amounts of other bacterial strains or species. Such compositions may be a culture that is substantially free from other species of organism. The invention may provide a composition comprising one or more strains from the species Enterococcus gallinarum, which does not contain bacteria from any other species or which comprises only de minimis or biologically irrelevant amounts of bacteria from another species for use in therapy.

The compositions of the invention can comprise more than one bacterial strain or species. For example, the compositions of the invention comprise more than one strain from within the same species (e.g. more than 1, 2, 3, 4, 5, 6, 7, 8, 9, 10, 15, 20, 25, 30, 35, 40 or 45 strains), and, optionally, do not contain bacteria from any other species. The compositions of the invention can comprise less than 50 strains from within the same species (e.g. less than 45, 40, 35, 30, 25, 20, 15, 12, 10, 9, 8, 7, 6, 5, 4 or 3 strains), and, optionally, do not contain bacteria from any other species. The compositions of the invention can comprise 1-40, 1-30, 1-20, 1-19, 1-18, 1-15, 1-10, 1-9, 1-8, 1-7, 1-6, 1-5, 1-4, 1-3, 1-2, 2-50, 2-40, 2-30, 2-20, 2-15, 2-10, 2-5, 6-30, 6-15, 16-25, or 31-50 strains from within the same species and, optionally, do not contain bacteria from any other species. The compositions of the invention can comprise more than one species from within the same genus (e.g. more than 1, 2, 3, 4, 5, 6, 7, 8, 9, 10, 12, 15, 17, 20, 23, 25, 30, 35 or 40 species), and, optionally, do not contain bacteria from any other genus The compositions of the invention can comprise less than 50 species from within the same genus (e.g. less than 50, 45, 40, 35, 30, 25, 20, 15, 12, 10, 8, 7, 6, 5, 4 or 3 species), and, optionally, do not contain bacteria from any other genus. the compositions of the invention comprise 1-50, 1-40, 1-30, 1-20, 1-15, 1-10, 1-9, 1-8, 1-7, 1-6, 1-5, 1-4, 1-3, 1-2, 2-50, 2-40, 2-30, 2-20, 2-15, 2-10, 2-5, 6-30, 6-15, 16-25, or 31-50 species from within the same genus and, optionally, do not contain bacteria from any other genus. The invention can comprises any combination of the foregoing.

The composition can comprise a microbial consortium. For example, the composition comprises the bacterial strain having a 16S rRNA sequence that is at least 95% identical to SEQ ID NO:2, for example, which is an Enterococcus gallinarum, as part of a microbial consortium. For example, the bacterial strain is present in combination with one or more (e.g. at least 2, 3, 4, 5, 10, 15 or 20) other bacterial strains from other genera with which it can live symbiotically in vivo in the intestine. For example, the composition comprises a bacterial strain having a 16S rRNA sequence that is at least 95% identical to SEQ ID NO:2, for example, which is an Enterococcus gallinarum, in combination with a bacterial strain from a different genus. The microbial consortium can comprises two or more bacterial strains obtained from a faeces sample of a single organism, e.g. a human. The microbial consortium in the composition may not found together in nature. For example, the microbial consortium may comprises bacterial strains obtained from faeces samples of at least two different organisms that could be from the same species, e.g. two different humans, e.g. two different human infants or an infant human and an adult human. The two organisms could also be from different species, for example the two organism are a human and a non-human mammal.

If the composition of the invention comprises more than one bacterial strain, species or genus, then the individual bacterial strains, species or genera may be administered separately, simultaneously or sequentially. For example the more than one bacterial strains, species or genera are stored separately but are mixed together prior to use.

The bacterial strain for use in the invention can be obtained from human infant, adolescent or adult faeces. If the composition of the invention comprises more than one bacterial strain, then all of the bacterial strains can be obtained from the human infant, adolescent or adult faeces. The bacteria may have been cultured subsequent to being obtained from the human infant faeces and being used in a composition of the invention.

The compositions for use in accordance with the invention may or may not require marketing approval.

Preferably, the composition of the invention comprises lyophilised bacteria. Lyophilisation of bacteria is a well-established procedure and relevant guidance is available in, for example, references [49-51].

The invention provides the above pharmaceutical composition, wherein said bacterial strain may be spray dried. Preferably, the invention provides the above pharmaceutical composition, wherein the bacterial strain is lyophilised or spray dried and wherein the bacteria is either live, viable, and/or capable of partially or totally colonising the intestine.

In some cases, the lyophilised or spray dried bacterial strain is reconstituted prior to administration. In some cases, the reconstitution is by use of a diluent described herein.

The compositions of the invention can comprise pharmaceutically acceptable excipients, diluents or carriers.

The compositions according to the invention may comprise: a bacterial strain as used in the invention; and a pharmaceutically acceptable excipient, carrier or diluent; wherein the bacterial strain is in an amount sufficient to increase the microbiota diversity in a subject when administered to a subject in need thereof

The invention can provide a pharmaceutical composition comprising: a bacterial strain as used in the invention; and a pharmaceutically acceptable excipient, carrier or diluent; wherein the bacterial strain is in an amount sufficient to treat a disorder when administered to a subject in need thereof; and wherein the disorder is a decrease in microbiota diversity and/or in the stability of the microbiota in a subject diagnosed with brain, breast, endometrium, ovarian, prostate or colon cancer.

The amount of the bacterial strain in the composition may be from about 1×10³ to about 1×10¹¹ colony forming units per gram with respect to a weight of the composition.

The composition may be administered at a dose of 1 g, 3 g, 5 g or 10 g.

The composition may be administered by a method selected from the group consisting of oral, rectal, subcutaneous, nasal, buccal, and sublingual.

The composition may comprise a carrier selected from the group consisting of lactose, starch, glucose, methyl cellulose, magnesium stearate, mannitol and sorbitol.

The composition may comprise a diluent selected from the group consisting of ethanol, glycerol and water.

The composition may comprise an excipient selected from the group consisting of starch, gelatin, glucose, anhydrous lactose, free-flow lactose, beta-lactose, corn sweetener, acacia, tragacanth, sodium alginate, carboxymethyl cellulose, polyethylene glycol, sodium oleate, sodium stearate, magnesium stearate, sodium benzoate, sodium acetate and sodium chloride.

The composition may further comprise at least one of a preservative, an antioxidant and a stabilizer. The preservative can be selected from the group consisting of sodium benzoate, sorbic acid and esters of p-hydroxybenzoic acid.

The composition may be stored in a sealed container at about 4° C. or about 25° C. The container may be placed in an atmosphere having 50% relative humidity, at least 80% of the bacterial strain as measured in colony forming units, remains after a period of at least about: 1 month, 3 months, 6 months, 1 year, 1.5 years, 2 years, 2.5 years or 3 years. The sealed container may be a sachet or bottle. The composition of the invention as described herein can also be provided in a syringe.

The composition may be provided as a pharmaceutical formulation. For example, the composition may be provided as a tablet or capsule, wherein optionally the capsule is a gelatine capsule (“gel-cap”).

The compositions of the invention can be administered orally. Oral administration may involve swallowing, so that the compound enters the gastrointestinal tract, and/or buccal, lingual, or sublingual administration by which the compound enters the blood stream directly from the mouth. Pharmaceutical formulations suitable for oral administration include solid plugs, solid microparticulates, semi-solid and liquid (including multiple phases or dispersed systems) such as tablets; soft or hard capsules containing multi- or nano-particulates, liquids (e.g. aqueous solutions), emulsions or powders; lozenges (including liquid-filled); chews; gels; fast dispersing dosage forms; films; ovules; sprays; and buccal/mucoadhesive patches.

The pharmaceutical formulation can be an enteric formulation, i.e. a gastro-resistant formulation (for example, resistant to gastric pH) that is suitable for delivery of the composition of the invention to the intestine by oral administration. Enteric formulations may be particularly useful when the bacteria or another component of the composition is acid-sensitive, e.g. prone to degradation under gastric conditions. The enteric formulation comprises an enteric coating and can be an enteric-coated dosage form. For example, the formulation may be an enteric-coated tablet or an enteric-coated capsule, or the like. The enteric coating may be a conventional enteric coating, for example, a conventional coating for a tablet, capsule, or the like for oral delivery. The formulation may comprise a film coating, for example, a thin film layer of an enteric polymer, e.g. an acid-insoluble polymer.

The enteric formulation can be intrinsically enteric, for example, gastro-resistant without the need for an enteric coating. Thus, the formulation is an enteric formulation that does not comprise an enteric coating and the formulation of the capsule can be made from a thermogelling material. The thermogelling material can be a cellulosic material, such as methylcellulose, hydroxymethylcellulose or hydroxypropylmethylcellulose (HPMC). The capsule can also comprise a shell that does not contain any film forming polymer. The shell can comprises hydroxypropylmethylcellulose and does not comprise any film forming polymer (e.g. see [52]). The formulation can be an intrinsically enteric capsule (for example, Vcaps® from Capsugel).

The formulation can be a soft capsule. Soft capsules are capsules which may, owing to additions of softeners, such as, for example, glycerol, sorbitol, maltitol and polyethylene glycols, present in the capsule shell, have a certain elasticity and softness. Soft capsules can be produced, for example, on the basis of gelatine or starch. Gelatine-based soft capsules are commercially available from various suppliers. Depending on the method of administration, such as, for example, orally or rectally, soft capsules can have various shapes, they can be, for example, round, oval, oblong or torpedo-shaped. Soft capsules can be produced by conventional processes, such as, for example, by the Scherer process, the Accogel process or the droplet or blowing process.

### Culturing Methods

The bacterial strains for use in the present invention can be cultured using standard microbiology techniques as detailed in, for example, references [53-55].

The solid or liquid medium used for culture may be YCFA agar or YCFA medium. YCFA medium may include (per 100 ml, approximate values): Casitone (1.0 g), yeast extract (0.25 g), NaHCO₃(0.4 g), cysteine (0.1 g), K₂HPO₄ (0.045 g), KH₂PO₄ (0.045 g), NaCl (0.09 g), (NH₄)₂SO₄ (0.09 g), MgSO₄·7H₂O(0.009 g), CaCl₂) (0.009 g), resazurin (0.1 mg), hemin (1 mg), biotin (1 μg), cobalamin (1 μg), p-aminobenzoic acid (3 μg), folic acid (5 μg), and pyridoxamine (15 μg).

### Bacterial Strains for Use in Vaccine Compositions

The inventors have identified that the bacterial strains of the invention are useful for increasing and/or stabilising the microbiota diversity in a subject. This is likely to be a result of the effect that the bacterial strains of the invention have on the host immune system. Therefore, the compositions of the invention, in addition to maintaining and/or improving the microbiota diversity of a subject may also advantageously have the effect of treating or preventing cancer, when administered as vaccine compositions. The bacterial strains of the invention can be viable and/or capable of partially or totally colonising the intestine. The bacterial strains of the invention may also be killed, inactivated or attenuated. The compositions for use in a vaccine may comprise a vaccine adjuvant and can be administered via injection, such as via subcutaneous injection.

### General

The practice of the present invention will employ, unless otherwise indicated, conventional methods of chemistry, biochemistry, molecular biology, immunology and pharmacology, within the skill of the art. Such techniques are explained fully in the literature. See, e.g., references [56] and [57-63], etc.

The term “comprising” encompasses “including” as well as “consisting” e.g. a composition “comprising” X may consist exclusively of X or may include something additional e.g. X+Y.

The term “about” in relation to a numerical value x is optional and means, for example, x±10%.

The word “substantially” does not exclude “completely” e.g. a composition which is “substantially free” from Y may be completely free from Y. Where necessary, the word “substantially” may be omitted from the definition of the invention.

Unless specifically stated, a process or method comprising numerous steps may comprise additional steps at the beginning or end of the method, or may comprise additional intervening steps. Also, steps may be combined, omitted or performed in an alternative order, if appropriate.

Various embodiments of the invention are described herein. It will be appreciated that the features specified in each embodiment may be combined with other specified features, to provide further embodiments. In particular, embodiments highlighted herein as being suitable, typical or preferred may be combined with each other (except when they are mutually exclusive).

### MODES FOR CARRYING OUT THE INVENTION

### Methodology

### Bacterial Strains

MRX518—Enterococcus gallinarum strain, NCIMB 42488
MRX0554—Enterococcus gallinarum strain, NCIMB 42761
MRX0858—Enterococcus caselliflavus strain
REF 10—Enterococcus gallinarum strain, DSM100110

### 16S Amplicon Sequencing

A Qiagen DNeasy Blood & Tissue Kit was used following the manufacturer's instructions, to extract microbial DNA from 0.2 g of frozen faecal samples from treated EMT6-mice at Day-14, Day 0 and Day 22.

Preparation and sequencing of the 16S rRNA gene amplicons was carried out using the 16S Sequencing Library Preparation Nextera protocol developed by Illumina (San Diego, Calif., USA). 50 ng of each of the DNA faecal extracts was amplified using PCR and primers targeting the V3/V4 variable region of the 16S rRNA gene. The products were purified and forward and reverse barcodes were attached by a second round of adapter PCR. The resulting PCR products were purified, quantified and equimolar amounts of each amplicon were then pooled before being sent for sequencing to the commercial Supplier GATC GmbH, on either the MiSeq (2×250 bp chemistry) or HiSeq (2×300 bp chemistry) platforms.

### Microbiome Composition Data Analysis (Post-Sequencing)

The raw sequence data was merged and trimmed using the flash methodology. This generates a single read from the read pairs and filters out low quality reads. The USEARCH pipeline methodology (version 8.1.1861_i86_linux64) was used to identify singleton sequences that are only represented by one read and to hide them from the OTU (Operational Taxonomic Unit) generating step. This is done due to the likelihood that these reads do not represent true biological variation but instead are due to technical variation. The UPARSE algorithm was then used to cluster the sequences into OTUs at 97% similarity. This generates a list of representative sequences which reflect the sequence variation within the dataset. These representative sequences were assigned to taxonomic level using the RDP classifier for phylum to family level and the APC associated SPINGO classifier was used for the genus and species level.

Chimeric sequences are sequences that originate from two or more biologically distinct transcripts. Chimeric sequences occur when two sequences combine to generate a new sequence due to annealing of the 16S sequences which share a high level of similarity, even when the origin of these sequences are from phylogenetically distinct origins. These chimeric sequences were removed using the UCHIME chimera removal algorithm with the Chimeraslayer reference database (downloaded: 9th September 2016). The USEARCH global alignment algorithm was then used to map all reads, including singletons onto the remaining OTU sequences, which now reflect the true taxonomic variation of the initial samples. Individual sequences were grouped into OTUs to give microbiome compositional information (abundance and diversity). These steps allowed the abundance of each taxa in each sample to be estimated.

### High-Level Data Analysis

Alpha diversity was investigated using 1) the Shannon diversity index, which represents the number of taxa (richness) and their relative abundances (evenness) within each sample and 2) the number of observed species per sample (richness), using the phyloseq library.

To establish whether there were significant differences in the global microbiome profiles between groups, Permutational MANOVA was performed on the dissimilarity matrix using the Adonis function in R. Boxplots were constructed using the strip chart and boxplot functions in the R statistical software.

A negative binomial statistical methodology (DESeq2 methodology) was used to identify taxonomic variables that were significantly differentially abundant within chosen comparisons. Raw P-values produced were adjusted for multiple testing using the Benjamini/Hochberg methodology.

### Example 1—Changes in Microbiota after Enterococcus gallinarum Treatment

### Summary

The effect of Enterococcus gallinarum and Enterococcus caselliflavus on the diversity and stability of microbiota was tested in a mouse model.

### Study Design

Mice from a breast cancer model (EMT6) (n=40) were treated with either bacterial strains MRX518, MRX0554, MRX0858 or REF 10 at a concentration of 2×10⁸; anti-CTLA4 at a concentration of 10 mL/kg. The mice were treated with the respective bacterial strain on Day-14, inoculated with tumour cells on Day 0. Anti-CTLA4 was administered on D13 of the study. Faecal samples were collected at three time points during the study, Day-14, Day 0 and Day 22 (end of the study). Across the whole study 120 faecal samples were collected.

No data were returned on 9 samples as they either failed to amplify pre-sequencing or the number of reads returned from sequencing was too low for analysis. In total data was returned for n=111 samples. Table 1 summarises the number of samples successfully returned for each treatment group at each time point.

TABLE 1
Number of samples for each treatment
group and time point where data were returned.

| | | | | | Anti- |
|---|---|---|---|---|---|
| Treatment | MRX518 | MRX0554 | MRX0858 | REF 10 | CTLA4 |
| Day -14 | n = 8 | n = 8 | n = 8 | n = 8 | n = 7 |
| (bacterization) | | | | | |
| Day 0 | n = 8 | n = 5 | n = 7 | n = 7 | n = 8 |
| (tumour cell | | | | | |
| inoculation) | | | | | |
| Day 22 | n = 7 | n = 8 | n = 6 | n = 8 | n = 8 |
| (end of study) | | | | | |

### Results

### Changes in Microbiota Diversity

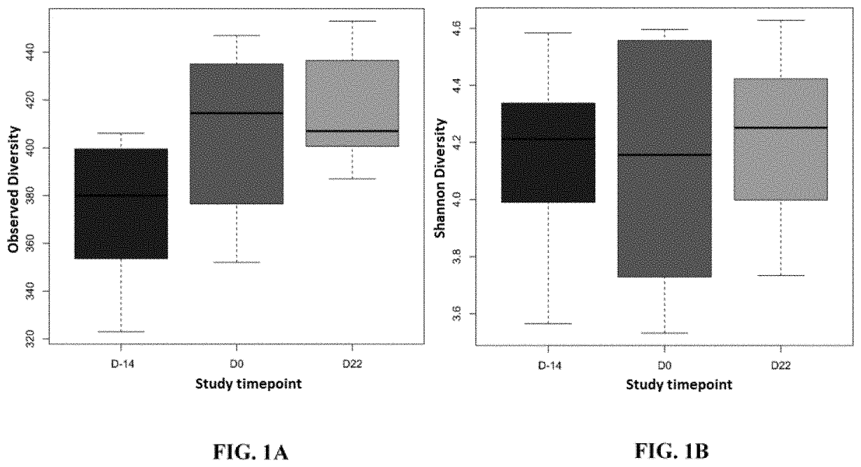

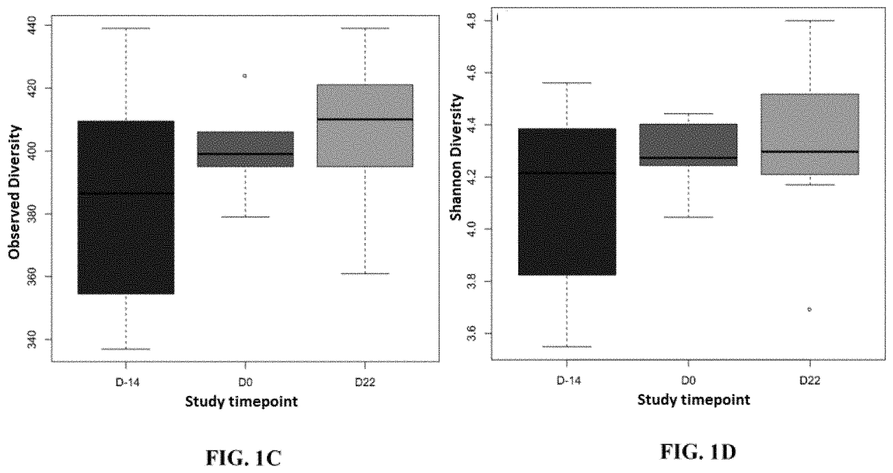

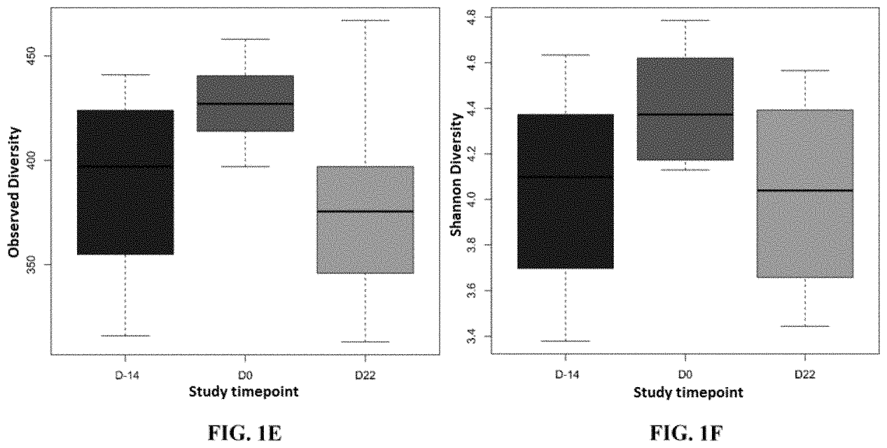

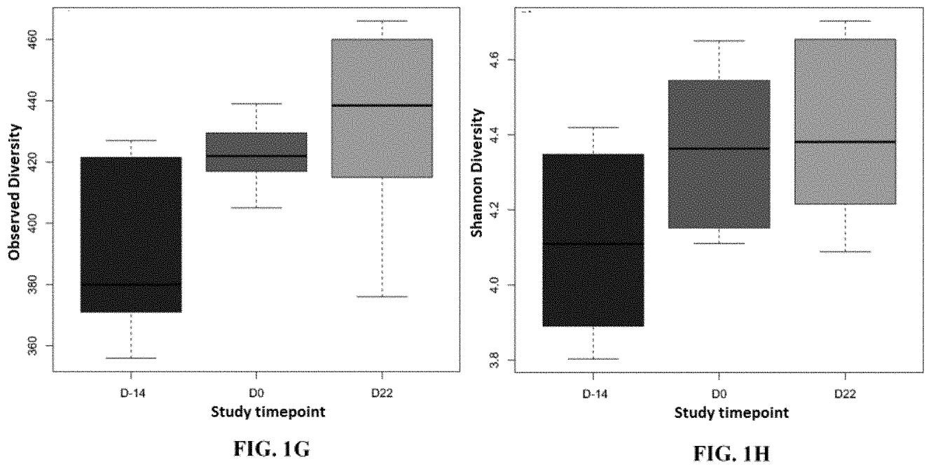

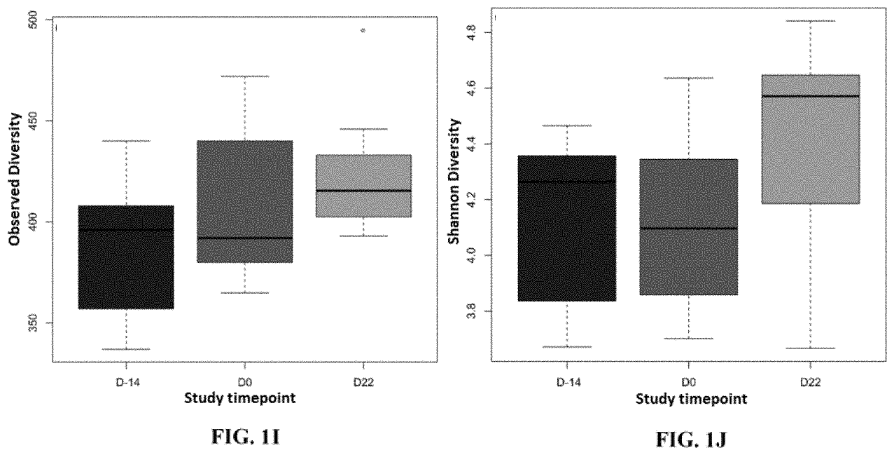

The effect of treatment on the microbiota diversity at Day-14, Day 0 and Day 22 can be seen in FIGS. 1a-j. These figures are consistent with an increase in diversity being observed after treatment with Enterococcus gallinarum or Enterococcus caselliflavus bacterial strains. For example, FIGS. 1a-d and FIGS. 1g-h show that the microbiome diversity increased in mice who were treated with Enterococcus gallinarum bacterial strains. FIGS. 1e-f show that treatment with Enterococcus caselliflavus can also increase the microbiome diversity in mice.

This increased diversity is maintained across the study, which is consistent with the ability of these bacterial strains to maintain the stability of the microbiome. For example, the microbiome diversity at Day 22 in FIGS. 1a-d and FIGS. 1e-f are similar to that observed at Day 0. This shows that Enterococcus gallinarum bacterial strains are able to maintain the stability of the microbiome.

### Variation in Taxa

The data disclosed in GB1712857.0 were reanalysed. Significantly differentially abundant taxa were observed in the treated groups at all three time points (Day-14, Day 0 and Day 22) when compared to a control group as shown below in Table 2 (p-value<0.05). ↑ indicates a significant increase of the taxa in the treated group and ↓ indicates a significant decrease of the taxa in the treated group. The quoted figures reflect the change in abundance (log 2 fold change). The number of differentially abundant taxa found in each treatment group at each time point are summarised in Table 3.

TABLE 2
Differentially abundant taxa found in each treatment group when
compared to a vehicle control at time points Day −14, Day 0 and Day 22 in EMT6 mice.

| | MRX518 | MRX554 | MRX858 | REF 10 | Anti-CTLA4 |
|---|---|---|---|---|---|
| Day | Eubacterium | Clostridium | 0 | 0 | Roseburia faecis |
| −14 | dolichum (↑, 3.565) | XIVa (↓, −4.599) | | | (↓, −3.98) |
| | | | | | Clostridium |
| | | | | | polysaccharolyticum |
| | | | | | m (↓, −5.131) |
| | | | | | Clostridium XIVa |
| | | | | | (↓ 4.806) |
| Day | Barnesiella | Clostridium | Roseburia (↑, 2.554) | 0 | 0 |
| 0 | intestinihominis (↓, −3.666) | XIVa (↓, −4.396) | Clostridium | | |
| | Oscillospira | | XIVa (↓, −2.227) | | |
| | guilliermondii (↑, 2.024) | | Lachnospiraceae | | |
| | Odoribacter | | (↑, 3.392) | | |
| | splanchnicus (↓, −3.094) | | Clostridium | | |
| | | | XIVa (↑, 4.676) | | |
| Day | Lachnospiraceae (↑, 3.483) | Clostridium | Clostridium | Clostridium | Clostridium XIVa |
| 22 | Clostridium XIVa (↓, −3.035) | XIVa (↓, −6.731) | XIVa (↑, 3.594) | XIVa (↑, 3.105) | (↓, −6.145) |
| | Firmicutes (↑, 4.253) | Clostridium | Clostridium | Eubacterium | Lachnospiraceae |
| | Lachnospiraceae (↑, 3.137) | XIVa (↓, −6.860) | XIVa (↓, −5.858) | (↓, −4.999) | (↑, 3.556) |
| | Clostridium XIVa (↓, −6.038) | Clostridium | Clostridium | Clostridium | Clostridium XIVa |
| | Eubacterium (↓, −2.373) | saccharolytic | XIVa (↓, −6.077) | XIVa (↓, −6.365) | (↓, −6.333) |
| | Clostridium XIVa (↓, −4.003) | um (↓, −4.884) | Clostridium | Lachnospiraceae | Clostridium |
| | Bacteroides | | saccharolyticum | (↓, −4.157) | saccharolyticum |
| | acidifaciens (↑, 2.880) | | (↓, −4.039) | Lachnospiraceae | (↓, −4.576) |
| | Barnesiella | | | (↑, 3.246) | |
| | intestinihominis (↓, −3.062) | | | Clostridium | |
| | | | | XIVa (↓, −4.213) | |

TABLE 3
Number of differentially abundant taxa found in
each treatment group between the study time points.

| | D-14 → D0 | D0 → D22 | D-14 → D22 |
|---|---|---|---|
| MRX518 | 13 | 13 | 17 |
| MRX0554 | 2 | 0 | 2 |
| MRX0858 | 13 | 0 | 11 |
| REF10 | 11 | 7 | 14 |
| Anti-CTLA4 | 1 | 9 | 31 |

After treatment with the bacterial strains E. gallinarum or E. caselliflavus the diversity of the microbiota increases and this increase includes increases in the abundance of Lachnospiraceae and Roseburia. One group of organisms for which there is a decrease in abundance is Clostridium XIVa Spp.

The data are consistent with the treatment E. gallinarum or E. caselliflavus leading to a more stabilised microbiome.

### Example 2—Changes in Microbiota Diversity after Enterococcus gallinarum or Enterococcus caselliflavus Treatment

### Summary

The effect of Enterococcus gallinarum or Enterococcus caselliflavus on the diversity and stability of microbiota was tested.

### Study Design

Mice from a lung cancer model (LLC) (n=48) were treated with either bacterial strains MRX518, MRX0554, MRX0858 of REF 10 at a concentration of 2×10⁸; anti-CTLA4 at a concentration of 10 mL/kg or were untreated. The mice were treated with the respective bacterial strain on Day-14, inoculated with tumour cells on Day 0, if relevant anti-CTLA4 was administered on D13 of the study. Faecal samples were collected at the end of the study on Day 18.

No data were returned on 11 samples as either the mice had died before the collection date or the sample failed to amplify pre-sequencing. In total data were returned for n=37 samples. Table 4 summarises the number of samples successfully returned for each treatment group at each time point.

TABLE 4:
Number of samples for each treatment group
and time point where data was returned.

| | | | | | | Anti- |
|---|---|---|---|---|---|---|
| | Untreated | MRX518 | MRX0554 | MRX0858 | REF10 | CTLA4 |
| D 18 (end of | n = 5 | n = 5 | n = 7 | n = 7 | n = 6 | n = 7 |
| treatment) | | | | | | |

### Results

### Changes in Microbiota Diversity

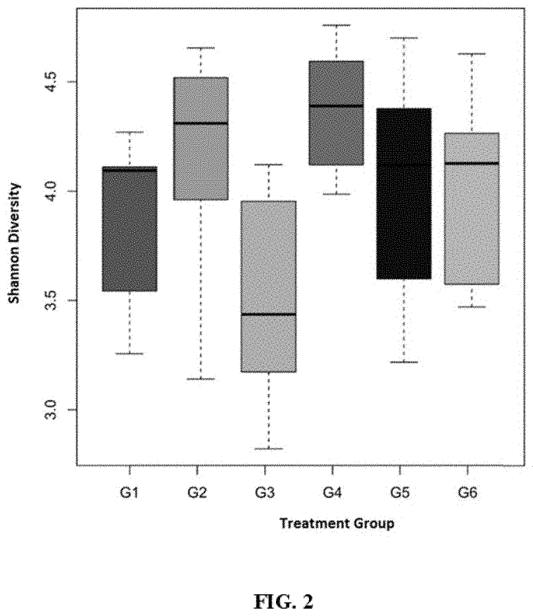

The effect of treatment on the change in microbiota diversity at Day 18 can be seen in FIG. 2. The Shannon diversity index increased after treatment with MRX518 and MRX0858 compared to the untreated control, which is consistent with the ability of E. gallinarum or E. caselliflavus bacterial strains to increase the diversity of the microbiota. A decrease in diversity was observed after treatment with MRX554. This data, however is likely due to experimental error, because FIGS. 1c-d show that this bacterial strain is capable of increasing the microbiome diversity.

### Variation in Taxa

The data disclosed in GB1712857.0 were reanalysed. DESeq2 analysis revealed differentially abundant taxa in treated groups when compared to a control group as shown in Table 5 below. ↑ indicates a significant increase of the taxa in the treated group and ↓ indicates a significant decrease of the taxa in the treated group. The quoted figures reflect the change in abundance (log 2 fold change).

TABLE 5
Number of differentially abundant taxa per treatment
group when compared to a vehicle control at D18.

| MRX518 | MRX554 | MRX858 | REF 10 | Anti-CTLA4 |
|---|---|---|---|---|
| Clostridium | Clostridium XIVa | Alistipes | Gordonibacter | Clostridium XIVa |
| disporicum (↓, −3.853) | (↑, 5.162) | massiliensis (↓, −6.215) | pamelaeae (↑, | (↓, −4.845) |
| Lactobacillus (↑, 2.356) | Barnesiella | Alistipes | 4.265) | Ruminococcaceae |
| Alloprevotella | intestinihominis | massiliensis (↓, −7.733) | | (↓, −3.044) |
| rava (↑, 2.397) | (↑, 4.180) | Barnesiella | | Clostridium |
| Barnesiella | Lachnospiracea | intestinihominis (↑, 6.473) | | disporicum (↓, −6.256) |
| intestinihominis | Incertae sedis (↑, 4.303) | Turicibacter | | Ruminococcus (↑, 3.049) |
| (↑, 7.134) | Barnesiella | sanguinis (↑, 3.081) | | Acetatifactor muris |
| Eubacterium (↑, 3.548) | intestinihominis | Gordonibacter | | (↑, 3.439) |
| Roseburia | (↑, 5.978) | pamelaeae (↑, 5.246) | | Roseburia |
| intestinalis (↓, −2.911) | Clostridium viride | Alistipes (↓, −4.532) | | intestinalis (↓, −5.368) |
| Eubacterium | (↓, −3.939) | | | Eubacterium |
| ramulus (↓, −2.430) | Gordonibacter | | | ramulus (↓, −2.571) |
| Barnesiella | pamelaeae (↑, 4.095) | | | Roseburia faecis (↑, 3.251) |
| intestinihominis | | | | Lachnospiraceae (↓, −2.699) |
| (↑, 3.392) | | | | Clostridium |
| Barnesiella | | | | lavalense (↓, −1.747) |
| intestinihominis | | | | Clostridium XIVa |
| (↑, 3.716) | | | | (↓, −4.036) |
| Barnesiella | | | | Clostridium viride |
| intestinihominis | | | | (↓, −3.991) |
| (↑, 3.157) | | | | Eubacterium |
| Barnesiella | | | | plexiclaudatum (↓, −3.199) |
| intestinihominis | | | | Barnesiella |
| (↑, 2.298) | | | | intestinihominis (↑, 2.443) |
| Gordonibacter | | | | Eubacterium |
| pamelaeae (↑, 4.528) | | | | plexicaudatum (↓, −2.976) |
| Akkermansia | | | | Clostridium XIVa |
| muciniphila (↑, 5.431) | | | | (↑, 4.097) |
| | | | | Turicibacter |
| | | | | sanguinis (↑, 3.371) |
| | | | | Olsenella profusa (↑, 3.197) |
| | | | | Clostridium XIVa |
| | | | | (↓, −3.626) |
| | | | | Eubacterium |
| | | | | siraeum (↑, 3.341) |

Treatment with the bacterial strains E. gallinarum or E. caselliflavus can increase the diversity of the microbiota, in particular the proportion of Lachnospiraceae and Roseburia (PMID: 26416813)

Clostridium Cluster XIVa spp., are decreased after the treatment with the bacterial strains E. gallinarum or E. caselliflavus.

Treatment further resulted in selective enrichment of Barnesiella intestinihominis. This strain has previously been shown to increase IFN-γ-producing γδT cells in tumours and induce specific-memory Th1 cell immune responses, thus providing an additional mechanism of immunostimulation [38]. Thus, when administered to subjects, the compositions of the present invention will not only function to stabilise and/or improve the microbiota diversity of those subjects, but also promote the growth of an organism demonstrated as having an anticancer effect, which is particularly advantageous if those subjects are cancer patients, or subjects identified as being at risk of cancer.

### Example 3—Stability Testing

A composition described herein containing at least one bacterial strain described herein is stored in a sealed container at 25° C. or 4° C. and the container is placed in an atmosphere having 30%, 40%, 50%, 60%, 70%, 75%, 80%, 90% or 95% relative humidity. After 1 month, 2 months, 3 months, 6 months, 1 year, 1.5 years, 2 years, 2.5 years or 3 years, at least 50%, 60%, 70%, 80% or 90% of the bacterial strain shall remain as measured in colony forming units determined by standard protocols.

### Example 4—Metabolomics Analysis

### Summary

The effect of Enterococcus gallinarum on the metabolomic profile of microbiota in mice was investigated.

### Study Design

Mice from a breast cancer model (EMT6) were treated with a concentration of 2×10⁸ in 200μl of MRX518 (n=9), MRx0518 (n=8), MRx0554 (n=8), MRx0858 (n=8), or REF10-DSM100110 (n=8) at; or received anti-CTLA4 (n=8) at a concentration of 10 mL/kg. Dosing with the bacterial species commenced on Day-14. The mice were engrafted with tumour cells on day DO. Treatment with anti-CTLA-4 antibody (10 mg/kg, IP) was initiated when tumours reached a volume of 50-70 mm³. Tumour volumes were measured every 3-4 days

Caecal content was isolated from snap frozen caecum and short chain fatty acids, polar and lipid metabolites extracted separately from the faecal slurry. The extracts were analysed by GC-MS (short chain fatty acids) and LC-MS analysis using HILIC (polar metabolites) and UPLC (lipid metabolites). Data was analysed through statistical t-tests with Benjamini-Hochberg correction at a false discovery rate of 10%, in order to compare the untreated group to all other groups. Metabolites were statistically significant if the p value was less than the Benjamini-Hochberg critical value calculated for each compound (i/m)Q.

Mice treated with MRX518 had significant differences in their metabolite profiles (Table 6). In contrast, anti-CTLA-4 treatment was not associated with any changes in metabolite production. Furthermore, metabolites along the whole pentose phosphate pathway are decreased during MRx0518 and MRx0554 treatment.

Mice treated with MRx0518 and MRx0554 also showed a decrease in members of the pentose phosphate pathway including ribose 5-phosphate, erythrose 4-phosphate and sedoheptulose 7-phosphate. As discussed above, this is advantageous as high expression levels of members of this pathway is associated with poor outcome in cancer patients and thus, when the compositions of the invention are administered to stabilise and/or enhance the microbiota stability of such patients, those compositions will additionally contribute to the prevention or treatment of cancer.

TABLE 6
Polar, SCFA and lipid metabolites that were significantly different between the MRx0518
group or anti-CTLA-4 group compared to the untreated control group.

| | Group 3 | Group 4 | Group 5 | Group 6 | Group 7 |
|---|---|---|---|---|---|
| | (MRx0518) | (MRx0554) | (MRx0858) | (REF10) | (Anti-CTLA4) |
| Polar | Neu5AC ↑↑ | ManNAc ↑ | ManNAc ↑ | ManNAc ↑ | None |
| Metabolites | Glutamine ↑ | Cytidine ↑ | Cytidine ↑ | Cytidine ↑ | |
| (positive | Serine ↑ | Serine ↑ | | Glutamine ↑ | |
| ionisation) | Plus 12 | Betaine ↑ | | Serine ↑ | |
| | unidentified | Neu5AC ↑ | | Neu5AC ↑ | |
| | metabolites | Glutamine ↑ | | Histidine ↑ | |
| | | (iso)leucine ↑ | | Plus 6 | |
| | | Histidine ↑ | | unidentified | |
| | | Proline ↑ | | metabolites | |
| | | Aspartic acid ↑ | | | |
| | | Citruline ↑ | | | |
| | | Plus 11 | | | |
| | | unidentified | | | |
| | | metabolites | | | |
| Polar | Glucose 1- | Glucose 1- | None | None | None |
| Metabolites | Phosphate ↓ | Phosphate ↓ | | | |
| (negative | Sedoheptulose- | Sedoheptulose-7- | | | |
| ionisation) | 7-phosphate ↓ | phosphate ↓ | | | |
| | Glucose 6- | Glucose 6- | | | |
| | Phosphate ↓ | Phosphate ↓ | | | |
| | Ribose 5- | Ribose 5- | | | |
| | Phosphate ↓ | Phosphate ↓ | | | |
| | Erythrose-4- | Erythrose-4- | | | |
| | phosphate ↓ | phosphate ↓ | | | |
| | Plus 145 | Pantothenic acid ↑ | | | |
| | unidentified | Plus 23 | | | |
| | metabolites | unidentified | | | |
| | | metabolites | | | |
| Short Chain | Butyrate ↓ | Butyrate ↓ | None | None | None |
| Fatty Acid | | | | | |
| Metabolites | | | | | |
| Lipid | 3 unidentified | None | 2 lipids | None | None |
| Metabolites | metabolites | | | | |
| (positive | | | | | |
| ionisation) | | | | | |
| Lipid | 68 lipids | 6 lipids | 2 lipids | None | None |
| Metabolites | | | | | |
| (negative | | | | | |
| ionisation) | | | | | |

### Example 5—Changes in Microbiota after Enterococcus gallinarum Treatment

### Study Design

Healthy female Balb/C (BALB/cByJ) mice (n=210), 5-7 weeks old, were obtained from CHARLES RIVER (L′Arbresles) and injected with EMT-6 tumour cells to create a model for breast cancer. The study was made up of 7 treatment groups with 10 mice in each group sampled at 3 timepoints (D-15, D-1 and D22). The treatment groups were:

| Group | Treatment |
|---|---|
| G1 | Untreated |
| G2 | YCFA |
| G3 | MRx0518 |
| G4 | Anti-PD1 |
| G5 | Anti-PD1+ MRx0518 |
| G6 | Anti-CTLA4 |
| G7 | Anti-CTLA4 + MRx0518 |

The mice were treated with 2×10⁸ MRX518 via oral gavage on Day-15. On day-1 (D-1) the mice were engrafted with EMT-6 tumour cells by a subcutaneous injection of 1×10⁶ EMT-6 cells in 200 μL RPMI 1640 into the right flank. Anti-CTLA4 (BE0131, Bioxcell) and PD-1 were administered on D13 of the study by injection into the peritoneal cavity of mice at a volume of 10 m1/kg adjusted to the most recent individual body weight of mice. Faecal samples were collected at three time points during the study, Day-15, Day-1 and Day 22 (end of the study).

### Generation of Microbiome Composition Data—16S Amplicon Sequencing

A Pre-processing of the data was accomplished using an adaptation of our in-house pipeline. The quality of the reads returned from sequencing was poor, meaning a stringent quality filtering step was added. The Trimmomatic tool (v0.36) was used for trimming and quality filtering. The first 16 and 20 base pairs were removed from the forward and reverse reads respectively, to remove primer sequences. Following this, leading or trailing base pairs falling below a Phred quality score of 25 were trimmed. Next, a sliding window of 4 was applied to the data of and if the reads in the window fell below an average Phred score of 22 the end of the read was removed. If, after the previous quality trimming steps, one or both of a read pair fell below 180 base pairs then the read pair was removed. After quality filtering, the next step was to merge the reads using the FLASH tool (v1.2.11). This generates a single read from the read pairs while filtering out read pairs that do not combine.

### Microbiome Composition Data Analysis (Post-Sequencing)

Taxonomic classification of the representative OTU sequences was performed using QIIME (v1.9.1) and SPINGO (v1.3). The QIIME assign_taxonomy.py script was used to assign taxonomy to genus level by reference to the RDP 11.2 database. The SPINGO tool was used to assign species level classification. Any classifications with a bootstrap confidence below 0.8 were assigned the label “unclassified”.

TABLE 7
Dataset statistics.

| | |
|---|---|
| Number of samples | 210 |
| Read depth per sample | 17871 (6884.056) |
| (mean (standard deviation)) | |
| Number of OTUs generated | 4181 |

1 sample, 89794_D_15, had very low sequence depth of 1,086. Therefore all samples of animal 89794 were removed from diversity calculations.

### High-Level Data Analysis

Analysis was performed using R v3.4.3 statistical software. Compositional dissimilarity of the dataset was investigated using the Bray-Curtis dissimilarity metric. The Bray-Curtis metric was calculated on proportion-normalised data using the vegdist function from the vegan package. Principal Coordinate Analysis (PCoA) ordination plots were created to visualise the effect of the different treatments on taxa composition. The PCoA plots were created using the made4 and ggplot2 packages. Permutational MANOVA statistical tests on the Bray-Curtis dissimilarity were performed using the adonis function from the vegan package.

### Results

The differential abundance of taxa was tested using the R package DESeq2. The full dataset was modelled in such a way that the treatment effects were compared to the YCFA control. The tests were performed at the OTU level and at each of 5 levels of taxonomic classification: Phylum, Class, Order, Family and Genus. Taxa were deemed to be significant with an adjusted p-value of less than 0.05. Only taxa with a log fold change of greater than 0.5 were deemed to be reliable. This was repeated at each timepoint. There were no significant taxa at D-15. Significant taxa for D-1 and D22 are shown in Table 8 and Table 9, respectively. A positive log fold change indicates a taxa is increased in the treatment group compared to the vehicle.

TABLE 8
Differentially abundant taxa observed in each treatment group when compared to YCFA (vehicle) at D −1.

| | | | Classification | | Log2 fold | St. | Adjusted |
|---|---|---|---|---|---|---|---|
| Level | Treatment | Taxa | Level | Classification | change | error | p-value |
| Genus | Anti-CTLA4 | Anaerotruncus | NA | NA | −1.290 | 0.38 | 0.028 |
| OTU_Table | MRx0518 | OTU_50 | Family | Lachnospiraceae | 4.780 | 1.13 | 0.043 |
| OTU_Table | MRx0518 | OTU_121 | Genus | Bamesiella | 2.120 | 0.44 | 0.004 |
| OTU_Table | Anti-PD1 | OTU_121 | Genus | Bamesiella | 2.237 | 0.48 | 0.014 |
| OTU_Table | Anti-PD1 | OTU_354 | Family | Lachnospiraceae | 5.245 | 1.17 | 0.014 |
| OTU_Table | Anti-CTLA4 | OTU_77 | Family | Lachnospiraceae | −24.844 | 3.91 | 8.21E−07 |
| OTU_Table | Anti-CTLA4 | OTU_79 | Class | Clostridia | −1.398 | 0.32 | 0.014 |
| OTU_Table | Anti-CTLA4 | OTU_121 | Genus | Bamesiella | 2.382 | 0.48 | 0.002 |
| OTU_Table | Anti-CTLA4 | OTU_186 | Family | Lachnospiraceae | 4.196 | 0.98 | 0.017 |

TABLE 9
Differentially abundant taxa observed in each treatment group when compared to YCFA (vehicle) at D 22.

| | | | Classification | | Log2 fold | St. | Adjusted |
|---|---|---|---|---|---|---|---|
| Level | Treatment | Taxa | Level | Classification | change | error | p-value |
| Phylum | Anti-PD1 | Deferribacteres | NA | NA | 1.556 | 0.559 | 0.048 |
| Phylum | Anti-CTLA4 | Deferribacteres | NA | NA | 1.470 | 0.559 | 0.039 |
| Phylum | Anti-CTLA4 | Tenericutes | NA | NA | −2.587 | 0.741 | 0.004 |
| Class | Anti-PD1 | Deferribacteres | NA | NA | 1.526 | 0.559 | 0.044 |
| Class | Anti-PD1 | unclassified | NA | NA | −1.004 | 0.313 | 0.019 |
| Class | Anti-CTLA4 | Mollicutes | NA | NA | −2.583 | 0.761 | 0.010 |
| Class | Anti-CTLA4 | unclassified | NA | NA | −0.954 | 0.313 | 0.016 |
| Order | Anti-PD1 | Anaeroplasmatales | NA | NA | −2.182 | 0.774 | 0.041 |
| Order | Anti-PD1 | unclassified | NA | NA | −1.198 | 0.365 | 0.017 |
| Order | Anti-CTLA4 | Anaeroplasmatales | NA | NA | −2.997 | 0.775 | 0.002 |
| Order | Anti-CTLA4 | unclassified | NA | NA | −1.286 | 0.365 | 0.004 |
| Family | Anti-PD1 | Anaeroplasmataceae | NA | NA | −2.232 | 0.764 | 0.043 |
| Family | Anti-PD1 | unclassified | NA | NA | −1.185 | 0.407 | 0.043 |
| Family | Anti-CTLA4 | Anaeroplasmataceae | NA | NA | −2.981 | 0.764 | 0.002 |
| Family | Anti-CTLA4 | Desulfovibrionaceae | NA | NA | 0.972 | 0.348 | 0.042 |
| Family | Anti-CTLA4 | unclassified | NA | NA | −1.200 | 0.407 | 0.039 |
| Genus | Anti-CTLA4 | Anaeroplasma | NA | NA | −3.042 | 0.774 | 0.004 |
| OTU_Table | MRx0518 | OTU_125 | Family | Lachnospiraceae | 8.098 | 1.825 | 0.012 |
| OTU_Table | MRx0518 | OTU_2853 | Genus | Alistipes | 2.062 | 0.469 | 0.012 |
| OTU_Table | Anti-CTLA4 | OTU_125 | Family | Lachnospiraceae | 9.871 | 2.022 | 0.002 |

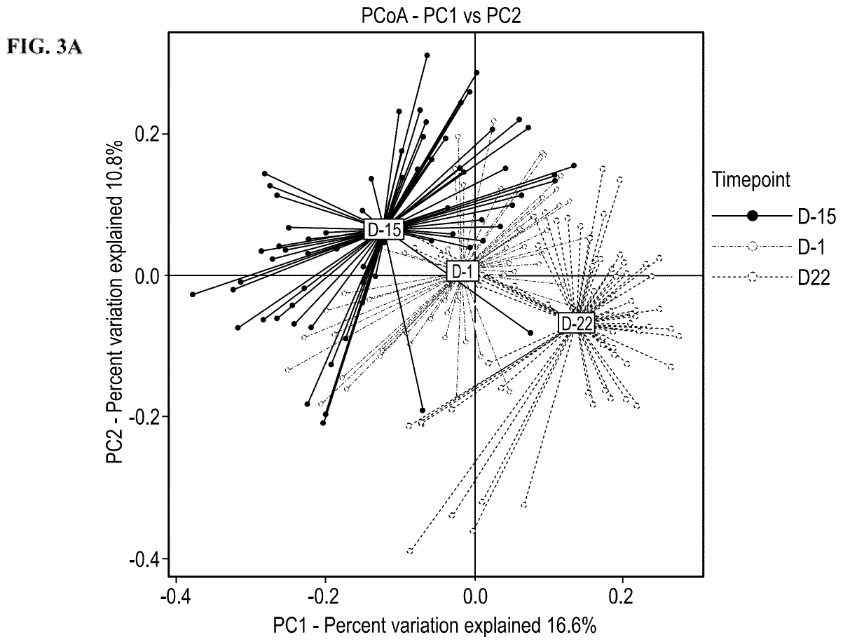

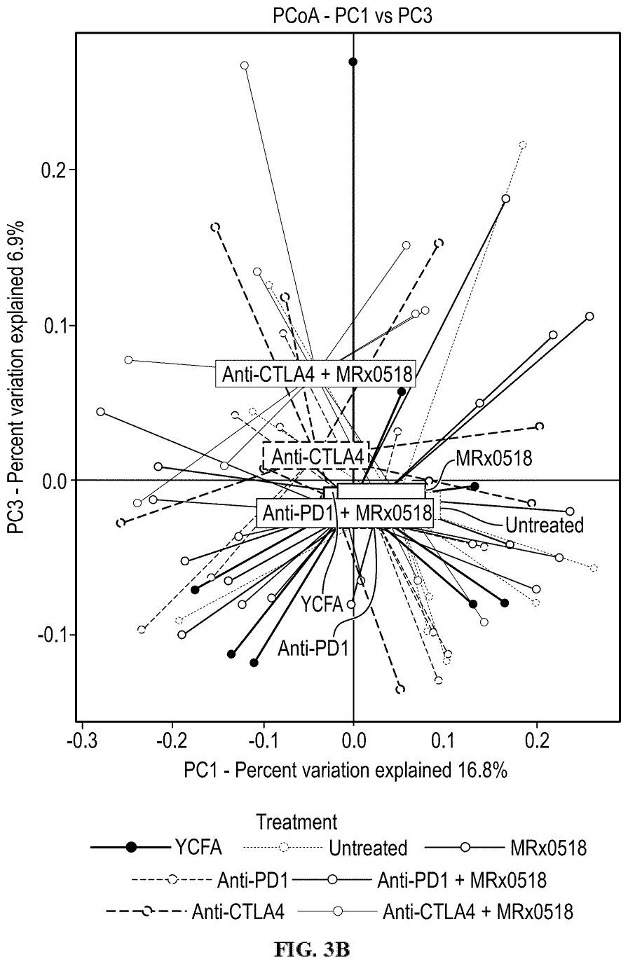

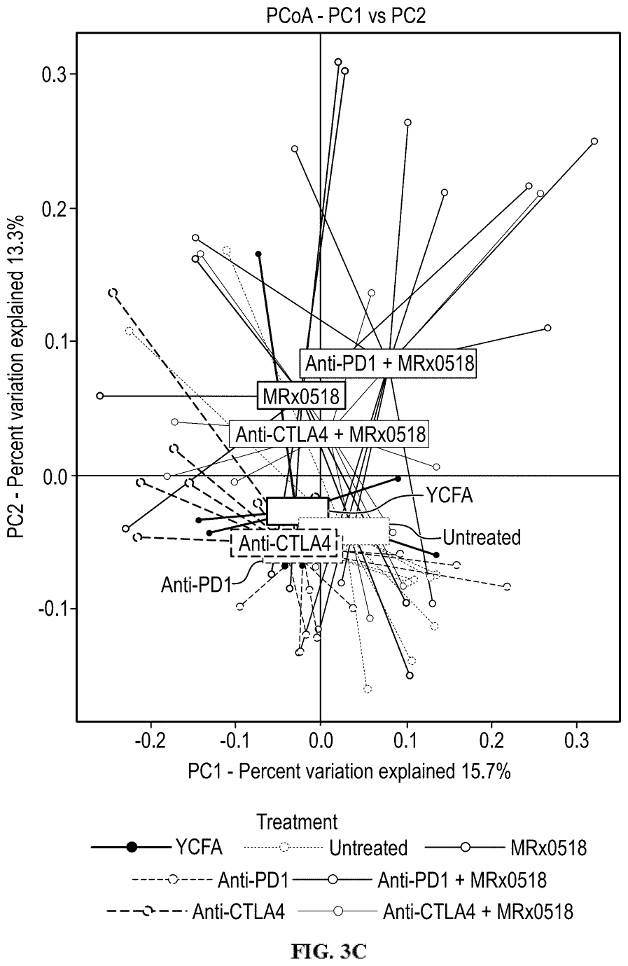

FIG. 3(c) shows a trend in the taxa associated with MRx0518 treatment. The mice treated with MRx0518 are clustered at the top of the coordination plot and have positive PC2 values, whereas the other treatments are clustered at the bottom of the coordination plot and have negative PC2 values. This is also shown in FIG. 3(a). FIG. 3(b) shows the taxa before treatment. It is evident that this clustering occurs only after treatment with MRx0518.

A complementary analysis was performed to identify taxa associated with the MRx0518 response as visualised on the second axis in the ordination plot in FIG. 3. This analysis shows the differences in taxa associated with the MRx0518 response observed at day 22.

This analysis consisted of a spearman correlation analysis as performed in R using the cor.test function. Only significant taxa are shown with a positive rho indicating that these taxa are associated with the trend and a negative rho indicating that the taxa are inversely associated.

Table 6 shows the taxa that are significantly different compared to the vehicle control (YFCA) at the D22 time point. These data confirm that treatment with MRx0518 leads to an increase in the abundance of Barnesiella species. The compositions of the invention are capable of increasing the levels of Barnesiella species, which have been implicated in cyclophosphamide efficacy.

TABLE 10
Taxa observed to be associated with MRx0518 treatment at D 22.

| | p.value | Rho |
|---|---|---|
| Alistipes | 1.77E−05 | −0.571 |
| Prevotella | 0.000332 | 0.491 |
| Parabacteroides | 0.00183 | 0.441 |
| Hallella | 0.00553 | 0.404 |
| Anaerotruncus | 0.0126 | 0.375 |
| Bacteroides | 0.0136 | 0.366 |
| Flavonifractor | 0.045 | 0.318 |
| Clostridium XIVb | 0.0451 | 0.308 |
| Vampirovibrio | 0.0451 | 0.313 |
| Anaeroplasma | 0.0487 | 0.301 |
| Barnesiella | 0.0487 | 0.297 |

Sequences

SEQ ID NO: 1 (Enterococcus gallinarum 16S rRNA gene - AF039900)

| | |
|---|---|
| 1 | taatacatgc aagtcgaacg ctttttcttt caccggagct tgctccaccg aaagaaaaag |
| 61 | agtggcgaac gggtgagtaa cacgtgggta acctgcccat cagaagggga taacacttgg |
| 121 | aaacaggtgc taataccgta taacactatt ttccgcatgg aagaaagttg aaaggcgctt |
| 181 | ttgcgtcact gatggatgga cccgcggtgc attagctagt tggtgaggta acggctcacc |
| 241 | aaggccacga tgcatagccg acctgagagg gtgatcggcc acactgggac tgagacacgg |
| 301 | cccagactcc tacgggaggc agcagtaggg aatcttcggc aatggacgaa agtctgaccg |
| 361 | agcaacgccg cgtgagtgaa gaaggttttc ggatcgtaaa actctgttgt tagagaagaa |
| 421 | caaggatgag agtagaacgt tcatcccttg acggtatcta accagaaagc cacggctaac |
| 481 | tacgtgccag cagccgcggt aatacgtagg tggcaagcgt tgtccggatt tattgggcgt |
| 541 | aaagcgagcg caggcggttt cttaagtctg atgtgaaagc ccccggctca accggggagg |
| 601 | gtcattggaa actgggagac ttgagtgcag aagaggagag tggaattcca tgtgtagcgg |
| 661 | tgaaatgcgt agatatatgg aggaacacca gtggcgaagg cggctctctg gtctgtaact |
| 721 | gacgctgagg ctcgaaagcg tggggagcga acaggattag ataccctggt agtccacgcc |
| 781 | gtaaacgatg agtgctaagt gttggagggt ttccgccctt cagtgctgca gcaaacgcat |
| 841 | taagcactcc gcctggggag tacgaccgca aggttgaaac tcaaaggaat tgacgggggc |
| 901 | ccgcacaagc ggtggagcat gtggtttaat tcgaagcaac gcgaagaacc ttaccaggtc |
| 961 | ttgacatcct ttgaccactc tagagataga gcttcccctt cgggggcaaa gtgacaggtg |
| 1021 | gtgcatggtt gtcgtcagct cgtgtcgtga gatgttgggt taagtcccgc aacgagcgca |
| 1081 | acccttattg ttagttgcca tcatttagtt gggcactcta gcgagactgc cggtgacaaa |
| 1141 | ccggaggaag gtggggatga cgtcaaatca tcatgcccct tatgacctgg gctacacacg |
| 1201 | tgctacaatg ggaagtacaa cgagttgcga agtcgcgagg ctaagctaat ctcttaaagc |
| 1261 | ttctctcagt tcggattgta ggctgcaact cgcctacatg aagccggaat cgctagtaat |
| 1321 | cgcggatcag cacgccgcgg tgaatacgtt cccgggcctt gtacacaccg cccgtcacac |
| 1381 | cacgagagtt tgtaacaccc gaagtcggtg aggtaacctt tttggagcca gccgcctaag |
| 1441 | gtgggataga tgattggggt gaagtcgtaa caaggtagcc gtatcggaag gtgcggctgg |
| 1501 | atcacc |

SEQ ID NO: 2 (consensus 16S rRNA sequence for Enterococcus gallinarum strain MRX518)
TGCTATACATGCAGTCGAACGCTTTTTCTTTCACCGGAGCTTGCTCCACCGAAAGAAAAAGAGTGGCGAACGGGTGA
GTAACACGTGGGTAACCTGCCCATCAGAAGGGGATAACACTTGGAAACAGGTGCTAATACCGTATAACACTATTTTC
CGCATGGAAGAAAGTTGAAAGGCGCTTTTGCGTCACTGATGGATGGACCCGCGGTGCATTAGCTAGTTGGTGAGGTA
ACGGCTCACCAAGGCCACGATGCATAGCCGACCTGAGAGGGTGATCGGCCACACTGGGACTGAGACACGGCCCAGAC
TCCTACGGGAGGCAGCAGTAGGGAATCTTCGGCAATGGACGAAAGTCTGACCGAGCAACGCCGCGTGAGTGAAGAAG
GTTTTCGGATCGTAAAACTCTGTTGTTAGAGAAGAACAAGGATGAGAGTAGAACGTTCATCCCTTGACGGTATCTAA
CCAGAAAGCCACGGCTAACTACGTGCCAGCAGCCGCGGTAATACGTAGGTGGCAAGCGTTGTCCGGATTTATTGGGC
GTAAAGCGAGCGCAGGCGGTTTCTTAAGTCTGATGTGAAAGCCCCCGGCTCAACCGGGGAGGGTCATTGGAAACTGG
GAGACTTGAGTGCAGAAGAGGAGAGTGGAATTCCATGTGTAGCGGTGAAATGCGTAGATATATGGAGGAACACCAGT
GGCGAAGGCGGCTCTCTGGTCTGTAACTGACGCTGAGGCTCGAAAGCGTGGGGAGCGAACAGGATTAGATACCCTGG
TAGTCCACGCCGTAAACGATGAGTGCTAAGTGTTGGAGGGTTTCCGCCCTTCAGTGCTGCAGCAAACGCATTAAGCA
CTCCGCCTGGGGAGTACGACCGCAAGGTTGAAACTCAAAGGAATTGACGGGGGCCCGCACAAGCGGTGGAGCATGTG
GTTTAATTCGAAGCAACGCGAAGAACCTTACCAGGTCTTGACATCCTTTGACCACTCTAGAGATAGAGCTTCCCCTT
CGGGGGCAAAGTGACAGGTGGTGCATGGTTGTCGTCAGCTCGTGTCGTGAGATGTTGGGTTAAGTCCCGCAACGAGC
GCAACCCTTATTGTTAGTTGCCATCATTTAGTTGGGCACTCTAGCGAGACTGCCGGTGACAAACCGGAGGAAGGTGG
GGATGACGTCAAATCATCATGCCCCTTATGACCTGGGCTACACACGTGCTACAATGGGAAGTACAACGAGTTGCGAA
GTCGCGAGGCTAAGCTAATCTCTTAAAGCTTCTCTCAGTTCGGATTGTAGGCTGCAACTCGCCTACATGAAGCCGGA
ATCGCTAGTAATCGCGGATCAGCACGCCGCGGTGAATACGTTCCCGGGCCTTGTACACACCGCCCGTCACACCACGA
GAGTTTGTAACACCCGAAGTCGGTGAGGTAACCTTTTTGGAGCCAGCCGCCTAAGGTG
SEQ ID NO: 3 (strain MRX518 chromosome sequence) - see electronic sequence listing.
SEQ ID NO: 4 (strain MRX518 plasmid sequence) - see electronic sequence listing.
SEQ ID NO: 5 (consensus 16S rRNA sequence for Enterococcus gallinarum strain MRx0554)
TTCACCGCGGCGTGCTGATCCGCGATTACTAGCGATTCCGGCTTCATGTAGGCGAGTTGCAGCCTACA
ATCCGAACTGAGAGAAGCTTTAAGAGATTAGCTTAGCCTCGCGACTTCGCAACTCGTTGTACTTCCCAT
TGTAGCACGTGTGTAGCCCAGGTCATAAGGGGCATGATGATTTGACGTCATCCCCACCTTCCTCCGGTT
TGTCACCGGCAGTCTCGCTAGAGTGCCCAACTAAATGATGGCAACTAACAATAAGGGTTGCGCTCGTT
GCGGGACTTAACCCAACATCTCACGACACGAGCTGACGACAACCATGCACCACCTGTCACTTTGCCCC
CGAAGGGGAAGCTCTATCTCTAGAGTGGTCAAAGGATGTCAAGACCTGGTAAGGTTCTTCGCGTTGCT
TCGAATTAAACCACATGCTCCACCGCTTGTGCGGGCCCCCGTCAATTCCTTTGAGTTTCAACCTTGCGG
TCGTACTCCCCAGGCGGAGTGCTTAATGCGTTTGCTGCAGCACTGAAGGGCGGAAACCCTCCAACACT
TAGCACTCATCGTTTACGGCGTGGACTACCAGGGTATCTAATCCTGTTCGCTCCCCACGCTTTCGAGCC
TCAGCGTCAGTTACAGACCAGAGAGCCGCCTTCGCCACTGGTGTTCCTCCATATATCTACGCATTTCAC
CGCTACACATGGAATTCCACTCTCCTCTTCTGCACTCAAGTCTCCCAGTTTCCAATGACCCTCCCCGGT
TGAGCCGGGGGCTTTCACATCAGACTTAAGAAACCGCCTGCGCTCGCTTTACGCCCAATAAATCCGGA
CAACGCTTGCCACCTACGTATTACCGCGGCTGCTGGCACGTAGTTAGCCGTGGCTTTCTGGTTAGATAC
CGTCAAGGGATGAACGTTCTACTCTCATCCTTGTTCTTCTCTAACAACAGAGTTTTACGATCCGAAAAC
CTTCTTCACTCACGCGGCGTTGCTCGGTCAGACTTTCGTCCATTGCCGAAGATTCCCTACTGCTGCCTC
CCGTAGGAGTCTGGGCCGTGTCTCAGTCCCAGTGTGGCCGATCACCCTCTCAGGTCGGCTATGCATCGT
GGCCTTGGTGAGCCGTTACCTCACCAACTAGCTAATGCACCGCGGGTCCATCCATCAGTGACGCAAAA
GCGCCTTTCAACTTTCTTCCATGCGGAAAATAGTGTTATACGGTATTAGCACCTGTTTCCAAGTGTTAT
CCCCTTCTGATGGGCAGGTTACCCACGTGTTACTCACCCGTTCGCCACTCTTTT

### REFERENCES

[1] Spor et al. (2011) Nat Rev Microbiol. 9(4):279-90.
[2] Eckburg et al. (2005) Science. 10; 308(5728):1635-8.
[3] Macpherson et al. (2001) Microbes Infect. 3(12):1021-35
[4] Macpherson et al. (2002) Cell Mol Life Sci. 59(12):2088-96.
[5] Mazmanian et al. (2005) Cell 15; 122(1):107-18.
[6] Frank et al. (2007) PNAS 104(34):13780-5.
[7] Scanlan et al. (2006) J Clin Microbiol. 44(11):3980-8.
[8] Kang et al. (2010) Inflamm Bowel Dis. 16(12):2034-42.
[9] Machiels et al. (2013) Gut. 63(8):1275-83.
[10] Sheflin et al (2014) Curr Oncol Rep. 16(10): 406.
[11] Lozupone (2012). Nature. 2012 Sep. 13; 489 (7415): 220-230
[12] DeSantis et al (2007)Microbial Ecology, 53, 3, 371-383.
[13] Vogt et al. (2017) Scientific Reports 7(1):13537 doi: 10.1038/s41598-017-13601-y
[14] Keshavarzian et al. (2015) Movement Disorders, 30, pp. 1351-1360 doi: 10.1002/mds.26307
[15] Kang et al. (2013) PLoS ONE 8(7) e68322 doi:10.1371/journal.pone.0068322
[16] Jangi et al. (2015) Nature Communications, 7, 12015 doi:10.1038/ncomms12015
[17] Chen et al. (2016) Sci. Rep. 6, 28484 doi:10.1038/srep28484
[18] Pozuelo et al. (2015) Scientific Reports. 5:12693. doi: 10.1038/srep12693
[19] Ott et al. (2004) Gut 53: 685-693 doi: 10.1136/gut.2003.025403
[20] Frank et al. (2007) Proc. Natl Acad. Sci. USA 104, 13780-13785 doi:
[21] Michail et al. (2012) Inflammatory Bowel Diseases 18(10) pp1799-1808. doi: 10.1002/ibd.22860
[22] Chen et al. (2016) Genome Med. 8:43 doi: 10.1186/s13073-016-0299-7
[23] Scher et al. (2015) Arthritis Rheumatol. 67, 128-139 doi: 10.1002/art.38892.
[24] Giongo et al. (2011) ISME J., 5 pp. 82-91 doi:10.1038/ismej.2010.92
[25] Claesson et al. (2012) Nature 488, 178-184. doi: 10.1038/nature11319
[26] Giloteaux et al. (2016) Microbiome, 4 (30) doi: 10.1186/s40168-016-0171-4
[27] Manero et al. (1999) Appl. Environ. Microbiol., 65, 10 4425-4430.
[28] Williams et al. (1990) Nucleic Acids Res. 18: 6531-6535.
[29] Collins et al. (1984) Int J Syst Evol Microbiol. 34: 220-223.
[30] Masco et al. (2003) Systematic and Applied Microbiology, 26:557-563.
[31] Current Protocols in Molecular Biology (F. M. Ausubel et al., eds., 1987) Supplement 30
[32] Smith & Waterman (1981) Adv. Appl. Math. 2: 482-489.
[33] Dayhoff et a/.(1978) Atlas of Protein Sequence and Structure, vol. 5, supp. 3
[34] Srůtková et al. (2011)J Microbiol. Methods, 87(1):10-6.
[35] Schenk et al (2011) Sic Signal, 4, 162
[36] Atarashi et al (2008) Nature, 455, 7214, 808-812
[37] Karmakar et al (2015) Nature Communications 7,10555
[38] Daillère et al (2016) Immunity. 18; 45(4):931-943
[39] WO2013/050792
[⁴⁰] McIlroy et al. (2018) Aliment Pharmacol Ther. 47:26-42.
[⁴¹1] Viaud et al. (2013) Science 342(6161): 971-976.
[⁴²1] Moustafa et al. (2018) Clinical and Translational Gastroenterology, 9, e132
[43] Cocco J (1987) Epidemiol Community Health.; 41(2): 89-93.
[44] Benito et al. (2017) Oncotarget.; 8(63):106693-106706
[45] Mitropoulou et al. (2013)J Nutr Metab. (2013) 716861.
[46] Kailasapathy et al. (2002) Curr Issues Intest Microbiol. 3(2):39-48.
[47] Handbook of Pharmaceutical Excipients, 2nd Edition, (1994), Edited by A Wade and P J Weller
[48] Remington's Pharmaceutical Sciences, Mack Publishing Co. (A. R. Gennaro edit. 1985)
[49] Miyamoto-Shinohara et al. (2008) J Gen. Appl. Microbiol., 54, 9-24.
[50] Cryopreservation and Freeze-Drying Protocols, ed. by Day and McLellan, Humana Press.
[51] Leslie et al. (1995) Appl. Environ. Microbiol. 61, 3592-3597.
[52] US 2016/0067188
[53] Handbook of Microbiological Media, Fourth Edition (2010) Ronald Atlas, CRC Press.
[54] Maintaining Cultures for Biotechnology and Industry (1996) Jennie C. Hunter-Cevera, Academic Press
[55] Strobel (2009) Methods Mol Biol. 581:247-61.
[56] Gennaro (2000) Remington: The Science and Practice of Pharmacy. 20th edition, ISBN: 0683306472.
[57] Molecular Biology Techniques: An Intensive Laboratory Course, (Ream et al., eds., 1998, Academic Press).
[58] Methods In Enzymology (S. Colowick and N. Kaplan, eds., Academic Press, Inc.)
[59] Handbook of Experimental Immunology, Vols. I-IV (D. M. Weir and C. C. Blackwell, eds, 1986, Blackwell Scientific Publications)
[60] Sambrook et al. (2001) Molecular Cloning: A Laboratory Manual, 3rd edition (Cold Spring Harbor Laboratory Press).
[61] Handbook of Surface and Colloidal Chemistry (Birdi, K. S. ed., CRC Press, 1997)
[62] Ausubel et al. (eds) (2002) Short protocols in molecular biology, 5th edition (Current Protocols).
[63] PCR (Introduction to Biotechniques Series), 2nd ed. (Newton & Graham eds., 1997, Springer Verlag)

1.-24. (canceled)

25. A method of increasing microbiome diversity in a gastrointestinal tract of a subject suffering from a reduced microbiome diversity relative to a healthy subject, as measured in feces, comprising administering to the subject a pharmaceutical composition containing an effective amount of an Enterococcus gallinarum or Enterococcus caselliflavus bacteria strain, wherein the administering results in an increase in an amount of Barnesiella bacteria in the gastrointestinal tract of the subject, relative to an amount of the Barnesiella bacteria prior to the administering.

26. The method of claim 25, wherein the Barnesiella bacteria is of species Barnesiella intestinihominis.

27. The method of claim 25, wherein the bacteria strain is of species Enterococcus gallinarum.

28. The method of claim 27, wherein the Enterococcus gallinarum bacteria strain comprises a 16s rRNA gene sequence with 95% sequence identity to SEQ ID NO:1, SEQ ID NO:2, or SEQ ID NO:5, as determined by a Smith-Waterman homology search algorithm using an affine gap search with a gap open penalty of 12, a gap extension penalty of 2, and a Blocks Substitution Matrix (BLOSUM) of 62.

29. The method of claim 27, wherein the Enterococcus gallinarum bacteria strain comprises a 16s rRNA gene sequence of SEQ ID NO:1, SEQ ID NO:2, or SEQ ID NO:5.

30. The method of claim 25, wherein the bacteria strain is of species Enterococcus caselliflavus.

31. The method of claim 25, wherein the administering results in an increase in Lachnospiraceae bacteria, relative to an amount prior to the administering.

32. The method of claim 25, wherein the administering results in an increase in Roseburia bacteria, relative to an amount prior to the administering.

33. The method of claim 25, wherein the bacteria strain is dried.

34. The method of claim 25, wherein the administering is oral.

35. The method of claim 25, wherein the pharmaceutical composition comprises from about 1×10³ to about 1×10¹¹ colony forming units (CFU)/g of the bacteria strain, with respect to a total weight of the pharmaceutical composition.

36. A method of increasing microbiome diversity in a gastrointestinal tract of a subject suffering from a reduced microbiome diversity relative to a healthy subject, as measured in feces, comprising administering to the subject a pharmaceutical composition containing an effective amount of an Enterococcus gallinarum or Enterococcus caselliflavus bacteria strain wherein the administering is sufficient to increase microbiome diversity in a gastrointestinal tract of a subject, as determined by an increase in a Shannon Diversity Index of at least 0.1 from a fecal sample obtained from the subject about 18 days after the administering, relative to a fecal sample obtained from the subject prior to the administering.

37. The method of claim 36, wherein the bacteria strain is of species Enterococcus gallinarum.

38. The method of claim 37, wherein the Enterococcus gallinarum bacteria strain comprises a 16s rRNA gene sequence with 95% sequence identity to SEQ ID NO:1, SEQ ID NO:2, or SEQ ID NO:5, as determined by a Smith-Waterman homology search algorithm using an affine gap search with a gap open penalty of 12, a gap extension penalty of 2, and a Blocks Substitution Matrix (BLOSUM) of 62.

39. The method of claim 37, wherein the Enterococcus gallinarum bacteria strain comprises a 16s rRNA gene sequence of SEQ ID NO:1, SEQ ID NO:2, or SEQ ID NO:5.

40. The method of claim 36, wherein the bacteria strain is of species Enterococcus caselliflavus.

41. The method of claim 36, wherein the bacteria strain is dried.

42. The method of claim 36, wherein the administering is oral.

43. The method of claim 36, wherein the administering is sufficient to increase the Shannon Diversity Index by about 0.2, relative to prior to the administering.

44. The method of claim 36, wherein the bacteria strain is encapsulated in a capsule.

45. The method of claim 36, wherein the pharmaceutical composition is an enteric formulation.

46. The method of claim 36, wherein the pharmaceutical composition comprises from about 1×10³ to about 1×10¹¹ colony forming units (CFU)/g of the bacteria strain, with respect to a total weight of the pharmaceutical composition.
